(19) United States
(12) Patent Application Publication
(10) Pub. No.: US 2026/0224497 A1
(43) Pub. Date: Aug. 6, 2026

(54) CONTROLLED RELEASE DELIVERY OF LOW WATER SOLUBILITY COMPOUNDS VIA ORAL ADMINISTRATION

(71) Applicant: Starton Therapeutics, Inc., Paramus, NJ (US)

(72) Inventors: Rod L. Hartwig, Sloatsburg, NY (US); Arturo Serrano-Batista, Riverdale, NJ (US); Jamie C. Oliver, Raleigh, NC (US)

(21) Appl. No.: 19/457,694

(22) Filed: Jan. 23, 2026

Related U.S. Application Data
(60) Provisional application No. 63/751,931, filed on Jan. 31, 2025.

(51) Int. Cl. A61K 9/48; A61K 9/00; A61K 9/20; A61K 31/438; A61K 31/567

(52) U.S. Cl. CPC A61K 9/485; A61K 9/0053; A61K 9/2009; A61K 9/2027; A61K 9/2054; A61K 9/2095; A61K 9/4833; A61K 9/4866; A61K 31/438; A61K 31/567

(57) ABSTRACT

A formulation for the controlled release of one or more active pharmaceutical ingredients (APIs) via oral administration is provided. The formulation includes an active pharmaceutical ingredient (API) having a water solubility of less than about 10 mg/mL and a melting point of greater than about 120° C. The API is present in the formulation in an amount ranging from about 0.05 wt. % to about 10 wt. %. The formulation also includes a carrier powder that is present in an amount ranging from about 0.5 wt. % to about 15 wt. %; a filler that is present in an amount ranging from about 15 wt. % to about 85 wt. %; and a polymer system comprising a combination of controlled release polymers. Further, the polymer system is present in an amount ranging from about 10 wt. % to about 70 wt. %. A method of making and a method of delivering the formulation via oral administration are also provided.

### RELATED APPLICATIONS

The present application claims priority to U.S. Provisional Patent Application No. 63/751,931, having a filing date of Jan. 31, 2025, the entire contents of which are incorporated herein by reference.

### TECHNICAL FIELD

The subject matter disclosed herein is generally directed to formulations and methods for the controlled release delivery of low water solubility active pharmaceutical ingredients (APIs). In particular, the formulations and methods described herein are for the oral administration of such APIs via a solid dosage form (e.g., tablet, capsule, pill, etc.).

### BACKGROUND

The primary obstacle in drug development is the solubility of the active pharmaceutical ingredient (API). Other important considerations include the stability of the API and the dosage form by which to deliver the API, such as via a parenteral, oral, transdermal or topical dosage form, including, but not limited to a patch, a solution, a suspension, an emulsion, a gel, a tablet, a capsule, a suppository, or any other dosage forms which must be selected to achieve an optimal delivery vehicle of a specified API. Once, these physicochemical characteristics and types of dosage forms are understood, the drug delivery profile becomes the priority for those skilled in the art of drug development.

As a typical rule of thumb in drug development, the more soluble the drug is, then the more amenable the drug is to absorption in the body. If the drug is insoluble in water or has very low solubility in water, which is the case for lenalidomide (LLD), pomalidomide (PMD), and dexamethasone, which have a water solubility of less than about 1 mg/mL at a neutral pH, it becomes difficult for absorption in the body to occur at an appreciable level. Thus, physical and chemical characterizations of the API in the presence of excipients are needed to assist in the solubility of the API in the developed formulation of a specified dosage form over the course of the delivery period so that the API can be absorbed by the body. In other words, if the API becomes more soluble upon administration, them the API is therefore more available for absorption to occur and at a specified interval of delivery to minimize or even mitigate possible Cₘₐₓ tolerability and toxicity events.

The typical drug products of lenalidomide (e.g., Revlimid®), pomalidomide (e.g., Pomalyst®), and dexamethasone (e.g., Hemady®) are delivered by oral administration of a tablet or capsule as an immediate release dosage form. For example, in the case of Revlimid® (lenalidomide capsules), the dissolution profile obtained by USP <711> and the FDA recommended dissolution method database exhibits no less than (NLT) 80% dissolution at 30 minutes for lenalidomide. Typical of immediate release dosage forms a standard specification is no less than (NLT) 85% (Q=80%+5%) dissolution in 60 minutes or less.

On the other hand, a molecule is typically less stable in a solubilized state, which presents it to be molecularly available by increased surface area for light, heat, hydrolysis, oxidation, or other possible degradation pathways. Thus, the complexity of producing a stable drug product is relative to the solubility and stability of a developed formulation.

Lenalidomide (LLD) is disclosed in literature as exhibiting very slight water solubility, as provided for in prescribing information for Revlimid®, where the solubility is indicated as being significantly lower in less acidic buffers, ranging from about 0.4 mg/mL to 0.5 mg/mL. Further, absolute bioavailability of lenalidomide is reported as 60%. The currently approved lenalidomide drug product, Revlimid®, is a solid oral dosage form presented as powder filled capsules in an immediate release dosage form in the range of 2.5 mg to 25 mg per capsule.

For lenalidomide, attempts have been made to improve the solubility of the API through the use of cocrystals, solvents, amorphous blends/mixtures, or other techniques to improve the bioavailability of the API, which is reported as low as 33%, which is beneficial in the production of immediate release dosage forms.

Pomalidomide (PMD) is disclosed in literature as exhibiting an even lower solubility than LLD, with very slight water solubility, as provided for in prescribing information for Pomalyst®, which states it has low solubility in all pH solutions, with a solubility of about 0.01 mg/mL. Absolute bioavailability of pomalidomide is reported as greater 70%. The currently approved pomalidomide drug product, Pomalyst®, is a solid oral dosage form presented as powder filled capsules in an immediate release dosage form in the range of 1 mg to 4 mg per capsule.

Dexamethasone (DEX) is disclosed in literature as exhibiting a very slight water solubility of less than 1 mg/mL. Absolute bioavailability of dexamethasone is reported as 60% to 80%. The currently approved dexamethasone drug products are generic ANDAs or a branded Hemady® are solid oral dosage form presented as immediate release tablet dosage forms. Most of the generic drug products comprising dexamethasone as tablets are an immediate release dosage form in the range of 0.5 mg up to about 10 mg per tablet.

With solid oral drug products, degradation pathways of hydrolysis or oxidation can be mitigated via the use of desiccants or oxygen scavengers, and/or by adjusting the formulation components. Further, utilizing a capsule shell that is formed as a hermetically-sealed uniform coating on the outside of the tablet or capsule can also act as a protective barrier to shield against oxygen or moisture.

In the oral drug product Revlimid®, specific pharmacokinetic (PK) profiles exhibit a high Cₘₐₓ within about 1 hour of initial delivery, as with most short half-life drugs, where T½ is less 5 hours, and an oral clearance of about 12 L/hr in the target patient population without repetitive dosing, where the blood levels then fall below a therapeutic threshold. Thus, the delivery of the LLD is not continuous or low dose, and these peaks (Cₘₐₓ) are typically associated with toxicity above therapeutic levels after administration and with valleys (Cₘᵢₙ) at subtherapeutic levels after about three (3) half-lives have elapsed. Further, repetitive dosing is not feasible as the drug toxicity is too great to overcome the risk-benefit relationship for dose management and patient outcomes of an immediate release oral drug product.

For Pomalyst®, the Cₘₐₓ is within about 2 to 3 hours with a reported half-life of about 7.5 hours and an oral clearance of about 6.5 to 10.8 L/hr.

In the oral drug products for dexamethasone, pharmacokinetic (PK) profiles exhibit a high Cₘₐₓ within about 1 to 2 hours of initial delivery, as with most short half-life, where T½ is less than about 7 hours, and an oral clearance of about 16 L/hr without repetitive dosing, where the blood levels then fall below a therapeutic threshold. Thus, the delivery of the API is not continuous or low dose, and these peaks (Cₘₐₓ) are typically associated with toxicity above therapeutic levels after administration and with valleys (Cₘᵢₙ) at subtherapeutic after about three (3) half-lives have elapsed. Further, repetitive dosing is not feasible as the drug toxicity is too great to overcome the risk-benefit relationship for dose management and patient outcomes of an immediate release oral drug product.

One of the primary analytical tests to assess an oral drug's release profile is by in vitro release testing (IVRT), also referred to as USP <711> Dissolution.

USP Apparatus 1 (Baskets) or USP Apparatus 2 (Paddles) are used frequently for assessment of oral dosage forms. USP Apparatus 2 (Paddles) is used, according to the FDA dissolution database, for Revlimid® (lenalidomide) capsules with 900 mL of 0.01 N HCl as the media, at 50 rpm with sample points of 10, 15, 20, 30 and 45 minutes, which support the dissolution profile for lenalidomide capsules, including Revlimid®, to be an immediate release (IR) dosage form in which no less than 80% of the drug content is released in less than 30 minutes.

USP Apparatus 2 (Paddles) is used, according to the FDA dissolution database, for Pomalyst® (pomalidomide) capsules with 900 mL of 0.1 N HCl as the media, at 50 rpm with sample points of 10, 15, 20, 30 and 45 minutes, which support the dissolution profile for pomalidomide capsules, including Pomalyst®, to be an immediate release (IR) dosage form in which no less than 80% of the drug content is released in less than 30 minutes.

Further, it was found in basic research in an article by Mahmoud (2019) that the dissolution profile of Revlimid® and a proposed generic formulation (ANDA) of LLD reported in four (4) different dissolution media was no less than 80% in 30 minutes and the no less than 80% level was actually achieved in all of the proposed dissolution media in less than 10 minutes, which supports the immediate release characteristics of the exiting dosage forms of oral tablets of LLD. This immediate release dosage form dissolution characteristic profile of no less than 80% being observed within 10 minutes is the primary reason for the high Cₘₐₓ value at about 1 hour after administration.

It is the immediate release of the 25 mg LLD which has this effect of such a high Cₘₐₓ reported of about 413 ng/mL. Also, due to the short half-life (about 3 hours to 5 hrs) of lenalidomide in humans, the drug is eliminated from the body and reaches subtherapeutic blood levels quickly.

Similarly, dexamethasone tablets do not have a specified dissolution method in the database and reference is made to the FDA dissolution guidance document (2018) for immediate release dosage forms. The USP references monograph USP <711> Dissolution with 500 mL of 1:100 dilute hydrochloric acid utilizing Apparatus 1 (Baskets) at 100 rpm. A time of dissolution of 45 minutes with no less than 70% released after 45 minutes supports that the tablets are in an immediate release dosage form.

The following tables provide evidence of the accepted dissolution profile of Revlimid® as compared to a developed generic formulation of lenalidomide capsules in the presence of four (4) different dissolution media from Mahmoud et al (2019), as well as the dissolution profiles of pomalidomide and dexamethasone.

TABLE 1
Dissolution Data of Lenalidomide and Revlimid ® 25 mg Capsules

| | | Lenalidomide | Revlimid ® % |
|---|---|---|---|
| Medium | Time (minutes) | % Released | Released |
| HCl 0.01N | 0 | 0 | 0 |
| | 10 | 92 | 91 |
| | 15 | 98 | 95 |
| | 20 | 98 | 95 |
| | 30 | 99 | 96 |
| | 45 | 99 | 96 |
| pH 1.2 | 0 | 0 | 0 |
| | 10 | 98 | 95 |
| | 15 | 99 | 98 |
| | 20 | 100 | 99 |
| | 30 | 100 | 99 |
| | 45 | 100 | 99 |
| pH 4.5 | 0 | 0 | 0 |
| | 10 | 91 | 98 |
| | 15 | 93 | 99 |
| | 20 | 93 | 100 |
| | 30 | 94 | 100 |
| | 45 | 94 | 100 |
| pH 6.5 | 0 | 0 | 0 |
| | 10 | 90 | 92 |
| | 15 | 91 | 94 |
| | 20 | 96 | 97 |
| | 30 | 97 | 99 |
| | 45 | 97 | 99 |

TABLE 2
Pharmacokinetic Data for Revlimid ® Immediate Release LLD

| Oral LLD | | | | | |
|---|---|---|---|---|---|
| Dose | | | Half-Life | Clear- | Calculated Cₘᵢₙ |
| (mg per | Cₘₐₓ | AUC_{0-inf} | (T½ in | ance | @ 24 hrs |
| day) | (ng/mL) | (ng*h/mL) | hrs) | (L/hr) | (ng/mL) |
| 25 mg | 413¹ | 1319 | 2.8 ± 0.4 | 19.1 | <1 |
| 25 mg | 568²* | 2091 | 3.3 | 11.9 | <1 |
| 25 mg | 390²** | 1369 | 2.8 | 18.1 | <1 |
| 20 mg | 302³ | 1035 | 3.4 | 19.3 | <1 |
| 10 mg | 160³ | 484 | 2.9 | 20.6 | <1 |
| 5.0 mg | 75³ | 207 | 2.7 | 24.1 | <1 |

¹Sourced from Chen et al (2012) as 25 mg oral suspension where clearance is about 19 L/hr.
²*Sourced from Chen et al (2017) in young health adults where clearance is about 18 L/hr.
²**Sourced from Chen et al (2017) in older adults where clearance is about 12 L/hr.
³Sourced from Starton Clinical Study Report of oral administration Revlimid ® STAR-LLD-CLL-001/SOS21427-21427X, where clearance is reported between 19 and 25 L/hr.

TABLE 3
Pharmacokinetic Data for Pomalyst ® Immediate Release PMD

| Oral PMD | | | Half-Life | Clear- | Calculated Cₘᵢₙ |
|---|---|---|---|---|---|
| Dose | Cₘₐₓ | AUC | (T½ in | ance | 24 hrs |
| (mg per day) | (ng/mL) | (ng*h/mL) | hrs) | (L/hr) | (ng/mL) |
| 5 mg¹ | 53.1 | 462 | 7.5 to 9.5 | 7-10 | <5 |
| 4 mg | 75 | 860 | 7.5 to 9.5 | 7.5² | <5 |
| 3 mg | NR | NR | 7.5 to 9.5 | 7-10 | <5 |
| 2 mg³ | 13 | 189 | 8.9 | 7-10 | <5 |
| 1 mg | NR | NR | 7.5 to 9.5 | 7-10 | <5 |

¹Sourced from Pomalyst ® prescribing information in patients with Kaposi sarcoma (KS)
²Sourced from Pomalyst ® prescribing information in patients with multiple myeloma (MM) or KS
³Sourced from Li-2015-Population PK of Pomalidomide as 2 mg in oral suspension
NR = Not Reported

TABLE 4
Pharmacokinetic Data for Hemady ® Immediate
Release Dexamethasone

| Oral DEX | | | | | Calculated |
|---|---|---|---|---|---|
| Dose | | | Half-Life | | Cₘᵢₙ @ |
| (mg per | Cₘₐₓ | AUC | (T½ in | Clearance | 24 hrs |
| day) | (ng/mL) | (ng*h/mL) | hrs) | (L/hr) | (ng/mL) |
| 20 mg¹ | 247 | 1271 | 4 | 15 | ~5 |
| 0.5-300 | NR | NR | 4 | 7.75-44.5 | <5 |
| mg¹ | | | | | |

¹Sourced from Hemady ® Clinical Pharmacology and Biopharmaceutics Review Application # 211379Orig1s000 submitted Sep. 6, 2018: Jun. 21, 2019.

Given the high Cₘₐₓ values and short dissolution times of immunomodulatory imide compounds and steroids with low water solubility as described above when in an immediate release dosage form, which increases toxicity risks initially upon dosing and then results in subtherapeutic levels of the APIs after just a few hours, a need currently exists for a formulation for oral drug delivery that is stable, soluble, and available in a controlled release dosage form to minimize side effects and improve the delivery profile to maximize patient benefit.

In other words, a formulation to support oral administration with a controlled release rate to achieve continuous and low dose delivery of immunomodulatory imide compounds, steroidal compounds, and other low water solubility APIs with short-half lives (e.g., less than about 8 hours) through a solid oral dosage form, such as a controlled release dosage form, as a tablet or capsule, as an improvement to the branded and generic versions of, for example, LLD and dexamethasone, would be beneficial. More specifically, a capsule or tablet where the API is maintained in a solid state, and, therefore, exhibits the best possible approach to achieving long-term stability in the formulation, would be useful.

### SUMMARY

Aspects and advantages of embodiments of the present disclosure will be set forth in part in the following description, or can be learned from the description, or can be learned through practice of the embodiments.

In one embodiment, a formulation for oral administration is provided. The formulation includes an active pharmaceutical ingredient (API) having a water solubility of less than about 10 mg/mL and a melting point of greater than about 120° C., wherein the API is present in the formulation in an amount ranging from about 0.05 wt. % to about 10 wt. % based on the total weight of the formulation; a carrier powder, wherein the carrier powder is present in the formulation in an amount ranging from about 0.5 wt. % to about 15 wt. % based on the total weight of the formulation; a filler, wherein the filler is present in the formulation in an amount ranging from about 15 wt. % to about 85 wt. % based on the total weight of the formulation; and a polymer system that includes one or more controlled release polymers, wherein the polymer system is present in the formulation in an amount ranging from about 10 wt. % to about 70 wt. % based on the total weight of the formulation.

In another aspect, the API can include an immunomodulatory imide compound, a Cereblon E3 ligase modulator (CELMoD), a steroid, or a combination thereof.

Further, the immunomodulatory imide compound can include lenalidomide, pomalidomide, iberdomide, or a combination thereof, the Cereblon E3 ligase modulator (CELMoD) can include mezigdomide, golcadomide, or a combination thereof, and the steroid can include dexamethasone, dexamethasone acetate, dexamethasone sodium phosphate, betamethasone, or a combination thereof.

In yet another aspect, the one or more APIs can include lenalidomide and dexamethasone.

In still another aspect, the one or more APIs an include pomalidomide and dexamethasone.

In an additional aspect, the carrier powder can include silicon dioxide, polyvinylpyrrolidone, or a combination thereof.

In another aspect, the carrier powder can be silicone dioxide, and the API can be present in the carrier powder at a weight ratio of the API to the carrier powder ranging from 1:1.25 to about 1:15.

In one more aspect, the carrier powder can include polyvinylpyrrolidone, and the API can be present in the carrier powder at a weight ratio of the API to the carrier powder ranging from about to about 1:0.5 to about 1:2.

In another aspect, the polymer system can include polyvinylpyrrolidone, hydroxypropyl cellulose, crosslinked polyacrylic acid, or a combination thereof.

In yet another aspect, the formulation can include a nonionic surfactant such as a poloxamer. Further, the poloxamer can be present in the formulation in an amount ranging from about 0.5 wt. % to about 15 wt. % based on the total weight of the formulation.

In still another aspect, the formulation can be surrounded by an enteric coating or is contained within an enteric capsule shell.

In an additional aspect, the API can include an immunomodulatory imide compound, and the API can exhibit a percent release in 0.01 N HCl ranging from about 1% to about 20% after about 10 minutes and a percent release in 0.01N HCl ranging from about 70% to about 100% after about 24 hours.

In another aspect, the API can include a steroid, and the API can exhibit a percent release in 0.01 N HCl ranging from about 0.5% to about 15% after about 15 minutes and a percent release in 0.01N HCl ranging from about 35% to about 100% after about 24 hours.

In one more embodiment, a method of making a formulation for oral administration is provided. The method includes: combining an active pharmaceutical ingredient (API) having a water solubility of less than about 10 mg/mL and a melting point of greater than about 120° C. with a carrier powder, a filler, and a polymer system comprising one or more controlled release polymers to form a dry powder blend.

In another aspect, the polymer system can include a polyvinylpyrrolidone, a hydroxypropyl cellulose, a crosslinked polyacrylic acid, or a combination thereof.

In still another aspect, the method can further include compressing the dry powder blend into tablet form.

In another embodiment, a method of delivering a formulation to a subject includes: administering the formulation to the subject orally. Further, the formulation includes: an active pharmaceutical ingredient (API) having a water solubility of less than about 10 mg/mL and a melting point of greater than about 120° C., wherein the API is present in the formulation in an amount ranging from about 0.05 wt. % to about 10 wt. % based on the total weight of the formulation; a carrier powder, wherein the carrier powder is present in the formulation in an amount ranging from about 0.5 wt. % to about 15 wt. % based on the total weight of the formulation; a filler, wherein the filler is present in the formulation in an amount ranging from about 15 wt. % to about 85 wt. % based on the total weight of the formulation; and a polymer system comprising one or more controlled release polymers, wherein the polymer system is present in the formulation in an amount ranging from about 10 wt. % to about 70 wt. % based on the total weight of the formulation.

In another aspect, the formulation can be administered via a tablet or capsule.

In yet another aspect, the formulation can be surrounded by an enteric coating or is contained within an enteric capsule shell.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

A full and enabling disclosure of the present disclosure to one skilled in the art, including the best mode thereof, is set forth more particularly in the remainder of the specification, including reference to the accompanying figures, in which:

FIG. 1 is a graph comparing the dissolution profiles of a lenalidomide formulation contemplated by the present disclosure compared to an immediate release formulation (Revlimid®);

FIG. 2 is a graph comparing the dissolution profiles of additional lenalidomide formulations contemplated by the present disclosure compared to an immediate release formulation (Revlimid®);

FIG. 3 is a graph comparing the dissolution profiles of additional lenalidomide formulations contemplated by the present disclosure compared to an immediate release formulation (Revlimid®);

FIG. 4 is a graph comparing the dissolution profiles of additional lenalidomide formulations contemplated by the present disclosure compared to an immediate release formulation (Revlimid®) and formulations without a combination of polymers contemplated by the present disclosure;

FIG. 5 is a graph illustrating blood levels of a single dose of lenalidomide in the form of an oral tablet of 5 mg, 10 mg, 20 mg, and 25 mg per day of Revlimid® over a time period of 24 hours (1 day);

FIG. 6 is a graph illustrating blood levels of three individual doses of lenalidomide in the form of an oral tablet of 5 mg, 10 mg, 20 mg, and 25 mg per day of Revlimid® over a time period of 72 hours (3 days);

FIG. 7 is a graph illustrating blood levels of a single dose of lenalidomide in the form of an oral tablet of 5 mg as contemplated by the present disclosure as compared to an oral tablet of 5 mg of Revlimid® over a time period of 24 hours (1 day);

FIG. 8 is a graph illustrating blood levels of three individual doses of lenalidomide in the form of an oral tablet of 5 mg as contemplated by the present disclosure as compared to an oral tablet of 5 mg of Revlimid® over a time period of 72 hours (3 days);

FIG. 9 is a graph comparing the dissolution profiles of two dexamethasone formulations contemplated by the present disclosure compared to an immediate release formulation (Hemady®);

FIG. 10 is a graph illustrating blood levels of a single dose of dexamethasone in the form of an oral tablet of 20 mg of Hemady® over a time period of 72 hours (3 days);

FIG. 11 is a graph illustrating blood levels of a single dose of dexamethasone in the form of an oral tablet of 5 mg as contemplated by the present disclosure as compared to an oral tablet of 5 mg of Hemady® over a time period of 24 hours (1 day);

FIG. 12 is a graph illustrating blood levels of a single dose of dexamethasone in the form of an oral tablet of 5 mg as contemplated by the present disclosure as compared to an oral tablet of 5 mg of Hemady® over a time period of 24 hours (1 day);

FIG. 13 is a graph illustrating blood levels of three individual doses of dexamethasone in the form of an oral tablet of 5 mg as contemplated by the present disclosure as compared to an oral tablet of 5 mg of Hemady® over a time period of 72 hours (3 days);

FIG. 14 is a graph comparing the dissolution profiles of additional lenalidomide formulations contemplated by the present disclosure compared to an immediate release formulation (Revlimid®);

FIG. 15 is a graph comparing the dissolution profiles of further lenalidomide formulations contemplated by the present disclosure compared to an immediate release formulation (Revlimid®);

FIG. 16 is a graph comparing the dissolution profiles of additional lenalidomide formulations contemplated by the present disclosure compared to an immediate release formulation (Revlimid®);

FIG. 17 is a graph comparing the dissolution profiles of additional lenalidomide formulations contemplated by the present disclosure compared to an immediate release formulation (Revlimid®);

FIG. 18 is a graph comparing the dissolution profiles of 1 wt. % and 2 wt. % lenalidomide formulation in an size 1, size 00 and size 000 HPMC capsule shells; and

FIG. 19 is a graph comparing the dissolution profiles of a 2 wt. % lenalidomide formulation in an enteric HPMC-P size 0 capsule shell compared to a HPMC size 0 capsule shell.

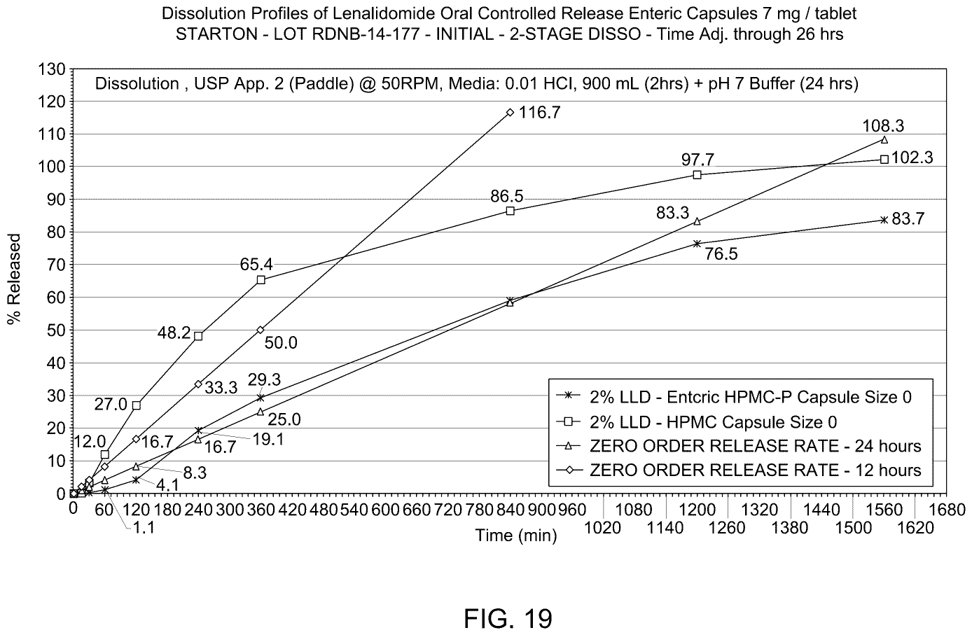

Repeat use of reference characters in the present specification and drawings is intended to represent the same or analogous features or elements of the present disclosure.

### DETAILED DESCRIPTION OF REPRESENTATIVE EMBODIMENTS

It is to be understood by one of ordinary skill in the art that the present discussion is a description of exemplary embodiments only, and is not intended as limiting the broader aspects of the present disclosure. Any of the features, components, or details of any of the arrangements or embodiments disclosed in this application are interchangeably combinable with any other features, components, or details of any of the arrangements or embodiments disclosed herein to form new arrangements and embodiments.

The present disclosure contemplates formulations and delivery methods for a controlled release rate of an oral administration of an immunomodulatory imide compound (e.g., lenalidomide, pomalidomide, or iberdomide), a steroidal compound (e.g., dexamethasone), or a combination thereof, in formulations which maintain the API in a solid state and maintain the stability of the API, while also achieving the desired release rate to achieve lower Cₘₐₓ levels (e.g., about 50%) and comparable exposure (total AUC) from about 60% less drug exposure based on existing formulations, resulting in better utilization of the administered API through continuous and low-dose release, leading to better absorption and efficiency of the available API, despite the short half-life of these APIs. Because the API is maintained in a solid state, it, therefore, exhibits the best possible approach to achieving long-term stability in the formulation, along with controlled release delivery.

In the case of short half-life drugs (e.g., APIs having a half-life of less than about 8 hours), the typical approach to achieving therapeutic blood levels is to perform multiple doses per day, (i.e., every 4 hours, every 6 hours, every 8 hours, or every 12 hours), which are common dosing regimens for many drugs, such as, by simple examples, acetaminophen, ibuprofen, Naproxen Sodium, decongestants, or antihistamines.

To achieve a controlled release oral dosage form for the APIs of the present disclosure, for example, as a tablet or capsule, it was initially hypothesized that equivalent drug loading would be needed to achieve bioavailability compared to the immediate release dosage forms. With this assumption, target drug loading was maintained to be the same. Surprisingly, however, the present inventors have found that a lower amount of API loaded into the tablet or capsule as compared to the immediate release drug products that are currently available is possible to achieve a bioavailable delivery profile for AUC exposure levels whilst at the same time minimizing Cₘₐₓ by more than about 50% and filling in the Cₘᵢₙ valleys associated with once a day dosing and short half-life drugs. This allows for a controlled release profile with a continuous low-dose release of the APIs from the tablet or capsule, such that the body is only exposed to limited amounts of the APIs over time of administration, such as a controlled release rate over a time frame of up to about 24 hours, such as over a time period of about 10 minutes, 15 minutes, 20 minutes, 30 minutes, 45 minutes, 1 hour, 2 hours, 3 hours, 4 hours, 5 hours, 6 hours, 8 hours, 10 hours, 12 hours, 18 hours, or 24 hours, or other justified dosing regimens where it is still considered a once a day dosage form for oral administration, but with controlled release instead of immediate release, which is the current state of the art.

Surprisingly, with the formulations of the present disclosure, it was found that a reproducible controlled release profile is possible to achieve a continuous low dose release profile in a near zero order profile for oral administration in the form of tablets of dry powder blends that may also be loaded into a capsule made from gelatin or hydroxypropyl methylcellulose (HPMC).

Generally speaking, the present disclosure is directed to a formulation for a stable solid oral dosage form for controlled release of an API having low water solubility, such as less than about 10 mg/mL, such as less than about 5 mg/mL, such as less than about 2.5 mg/mL, such as less than about 2 mg/mL, such as less than about 1.5 mg/mL, and/or a high melting point, such as about greater than about 120° C., such as greater than about 150° C., such as greater than about 200° C., such as greater than about 250° C. For instance, the API can include an immunomodulatory imide compound, a steroidal agent, or a combination thereof.

The present disclosure contemplates the use of a solid oral formulation that is substantially free of added water, to deliver an immunomodulatory imide drug (IMiD) compound, such as thalidomide, pomalidomide, lenalidomide, iberdomide, or a combination thereof, and/or one or more steroidal compounds, or a combination thereof, the specifics of which are discussed in more detail below. Other glucocorticoids with similar properties include betamethasone, prednisone, and methylprednisolone are contemplated for formulations as individual drugs or in combinations of one or more immunomodulatory imide compounds with one or more steroidal agents, such as a specified glucocorticoid. For example, dexamethasone combined with lenalidomide or dexamethasone combined with pomalidomide, or dexamethasone combined with iberdomide, are contemplated by the present disclosure.

The formulation of the tablet or capsule provides an increased level of dissolution control to provide controlled release of the drug throughout the gastrointestinal tract, which improves solubility, and the formulation includes an active pharmaceutical ingredient (API), a controlled release polymer, such as carbomer, povidone (PVP), hydroxypropyl cellulose (HPC), or a combination thereof; a slip agent, such as a nonionic surfactant, which can include polyoxyethylene (PEO); a filler, such as lactose, microcrystalline cellulose, or a combination thereof; a carrier powder for the API, such as silicon dioxide, PVP, or a combination thereof; and a glidant, such as magnesium stearate, sodium stearyl, or a combination thereof. Further, as stated above, the formulations are substantially free of added water.

The formulation is prepared as a dry powder blend for direct compression or powder fill into a capsule for oral administration. Not limited by over-simplification of the science in the formulation, it is contemplated that other processes to support formulation such as but not limited to roller compaction, wet or dry granulation, extrusion/spheronization, spray-drying, fluid bed coating, or other modalities to prepare a powder for tableting or encapsulation is considered. Further, the method of use requires the achievement of estimated doses of LLD of about 1 mg/day to about 25 mg/day of PMD of about 0.5 mg/day to about 5 mg/day, and of dexamethasone of about 5 mg/day to about 20 mg/day.

Without intending to be limited by any particular theory, the present inventors have found that the specific components of the formulation make it amenable to controlled, steady dissolution profiles to solve many problems associated with short half-life APIs. For instance, in 0.01 N HCl, the immunomodulatory imide compounds used as APIs in the formulations of the present disclosure can have a percent release ranging from about 1% to about 20%, such as from about 1.5% to about 15%, such as from about 2% to about 10% after 10 minutes; from about 45% to about 90%, such as from about 50% to about 87.5%, such as from about 55% to about 85% after about 12 hours; and from about 70% to about 100%, such as from about 75% to about 99%, such as from about 80% to about 98% after about 24 hours. Meanwhile, in 0.01 N HCl, the steroidal agents used as APIs in the formulations of the present disclosure can have a percent release ranging from about 0.5% to about 15%, such as from about 0.75% to about 10%, such as from about 1% to about 7.5% after about 10 minutes; from about 20% to about 40%, such as from about 22.5% to about 35%, such as from about 25% to about 30% after about 12 hours; and from about 35% to about 100%, such as from about 37.5% to about 95%, such as from about 40% to about 70% after about 24 hours. This is in stark contrast to existing immediate release lenalidomide and dexamethasone APIs, which exhibit greater than about 90% release in the first 10 to 15 minutes in 0.01 N HCl.

The various excipients in the formulations of the present disclosure described below have been chosen based on their commercial availability and inclusion in the FDA IIG raw materials list used for solid oral formulations to support tablet and/or capsule-based dosage forms within the maximum potency per unite dose and maximum daily exposure levels. The various components are discussed in more detail below.

API with Carrier Powder

The active pharmaceutical ingredients contemplated by the present disclosure include those APIs having low solubility in aqueous solutions. For instance, the API can have a water solubility of less than about 10 mg/mL, such as less than about 5 mg/mL, such as less than about 1.5 mg/mL, such as less than about 1.25 mg/mL, such as less than about 1 mg/mL. In some embodiments, the formulation of the present invention includes an API having a solubility in water ranging from about 0.001 mg/mL to about 2.0 mg/mL, such as from about 0.005 mg/mL to about 1.5 mg/mL, such as from about 0.01 mg/mL to about 1.0 mg/mL, and any ranges therebetween.

In one particular embodiment, the API can be an immunomodulatory imide compound. For instance, the immunomodulatory imide compound can include all pharmaceutically acceptable forms of an immunomodulatory imide compound, such as thalidomide, including analogs of thalidomide including lenalidomide (melting point of about 265° C. to about 270° C.), pomalidomide (melting point of about 318.5° C. to about 320.5° C.), and iberdomide including, for example, free base, salts, polymorphs, solvates, solutions, isomers, amorphous, crystalline, co crystalline, solid solution, prodrugs, analogs, derivatives, and metabolites and combinations thereof. The compound may be in the form of a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methane sulphonate, ethanesulphonate, benzenesulphonate, p-toluenesulphonate and pamoate salts. In one particular embodiment, the API can be lenalidomide. Further, the lenalidomide can be in the form of lenalidomide hemihydrate. In other embodiments, the lenalidomide can be an amorphous lenalidomide.

In another embodiment, the API can be a Cereblon (CRBN) E3 ligase modulators (CELMoD) agent. CELMoDs are a new drug class in myeloma. The emergence of CELMoDs is building on the well-established platform of immunomodulatory agents. Unlike immunomodulatory drugs like lenalidomide, CELMoDs exhibit enhanced specificity for transcription factors Ikaros (IKZF1) and Aiolos (IKZF3), which play key roles in multiple myeloma pathobiology. CELMoDs are generally oral (taken by mouth) medications that have many similarities to immunomodulatory agents. Yet, CELMoDs can be used even in patients who have relapsed after treatment with immunomodulatory agents. Mezigdomide is a CELMoD agent that is being studied in many clinical trials with different drug combinations and in different myeloma patient populations. Cereblon (CRBN) E3 ligase modulators (CELMoDs) are a new drug class used in the treatment of myeloma. While still in clinical trials, mezigdomide has demonstrated some positive results. Combination therapy with mezigdomide and dexamethasone (also called “MEZI-dex”) has also shown to be very effective in patients with at least 3 prior lines of therapy (meaning 1 or more complete). This includes some patients who have been treated with a B-cell maturation antigen (BCMA)-directed therapy. The overall response rates (ORRs) have been mostly in the 40% to 50% range. Cycles of a treatment regimen an include a single agent, a combination of several drugs, or a planned sequential therapy of various regimens. Mezigdomide has also been tested in combination with other agents earlier in the disease course, especially with proteasome inhibitors in patients who have had at least one prior line of therapy. ORR data were very encouraging, from 60% to 80%. Also, results from the CC-92480-MM-002 clinical trial of mezigdomide and dexamethasone in combination with either Darzalex or Empliciti showed promising efficacy and a manageable safety profile in patients with relapsed or refractory myeloma and 2 to 4 prior lines of therapy. Both mezigdomide and golcadomide have low solubilities in water, with both being nearly insoluble in water.

In another embodiment, the API can be a hormone such as a steroid. For example, the API can be a corticosteroid. Corticosteroids are a class of steroid hormones that are produced in the adrenal cortex of vertebrates, as well as the synthetic analogues of these hormones. Two main classes of corticosteroids, glucocorticoids, and mineralocorticoids, are involved in a wide range of physiological processes, including stress response, immune response, and regulation of inflammation, carbohydrate metabolism, protein catabolism, blood electrolyte levels, and behavior. Synthetic pharmaceutical drugs with corticosteroid-like effects are used in a variety of conditions, ranging from hematological neoplasms to brain tumors or skin diseases. Dexamethasone (melting point of about 262° C. to about 264° C.) and its derivatives are almost pure glucocorticoids, while prednisone and its derivatives have some mineralocorticoid action in addition to the glucocorticoid effect. Fludrocortisone is a synthetic mineralocorticoid. In any event, the steroid can be a corticosteroid comprising dexamethasone, dexamethasone acetate, dexamethasone sodium phosphate, betamethasone, or a combination thereof.

In some embodiments, the formulations contemplated by the present disclosure can include a combination of an immunomodulatory imide compound and a steroid, such as, but not limited to a combination of lenalidomide and dexamethasone or a combination of pomalidomide and dexamethasone.

Regardless of the particular API or APIs utilized, the amount of each API contained in the formulations contemplated by the present disclosure can range from about 0.05 wt. % to about 10 wt. %, such as from about 0.1 wt. % to about 7.5 wt. %, such as from about 0.2 wt. % to about 5 wt. %, or any ranges therebetween, based on the total weight of the formulation for oral administration, where it is to be understood that more than one API can be present (e.g., a combination of LLD and DEX, each being present individually in the weight percentages ranges described above).

### Carrier Powder for API

Regardless of the particular API utilized, the API can be loaded into a carrier powder. In one particular embodiment, the carrier powder can be silicon dioxide. The API can be loaded into the silicon dioxide by first dissolving the API into a solvent system comprising a polar aprotic solvent, such as n-methyl-2-pyrrolidone (NMP). Meanwhile, the silicon dioxide can be suspended in ethyl acetate, and the API-NMP solution can then be added into the silicon dioxide suspension. The blend is then mixed and subsequently tray dried to yield a dry powder with the desired ratio of API to silicon dioxide. The resulting dry powder is free of residual solvents to an acceptable ICH level for ethyl acetate and NMP. In one particular embodiment, the silicon dioxide can be utilized in conjunction with lenalidomide hemihydrate, although it is to be understood that the present disclosure also contemplates the use of silicon dioxide with any other immunomodulatory imide compound, steroid, or API of interest. Further, the API can be loaded into the silicon dioxide at a weight ratio of the API to the silicone dioxide ranging from about 1:1.25 to about 1:15, such as from about 1:1.5 to about 1:12.5, such as from about 1:2 to about 1:10, and any ranges therebetween.

In some embodiments, the silicone dioxide can be a mesoporous silicon dioxide that can have both internal and external porosity. The pores can have a diameter ranging from about 1 nanometer to about 50 nanometers, such as from about 2 nanometers to about 25 nanometers, such as from about 3 nanometers to about 10 nanometers. Meanwhile, the mesoporous silicon dioxide particles contemplated for use in the present disclosure can have a particle size ranging from about 1 micrometer to about 10 micrometers, such as from about 1.5 micrometers to about 9.5 micrometers, such as from about 2 micrometers to about 9 micrometers, which is larger than the particle size of fumed silica particles, which can, on average, have a 5 nanometer to 50 nanometer particle size, and colloidal silica particles, which can, on average, have a 2 nanometer to 100 nanometer particle size. Further, the average pore volume can range from about 0.2 cm³/gram to about 2 cm³/gram, such as from about 0.3 cm³/gram to about 1.9 cm³/gram, such as from about 0.4 cm³/gram to about 1.8 cm³/gram. As such, the silicon dioxide particles contemplated by the present disclosure can have an increased surface area compared to colloidal silicon dioxide and fumed silicon dioxide, both of which are generally non-porous. For example, the surface area of the mesoporous silicon dioxide particles can range from about 300 m²/g to about 700 m²/g, such as from about 350 m²/g to about 650 m²/g, such as from about 400 m²/g to about 600 m²/g, and any ranges therebetween. Without intending to be limited by any particular theory, the present inventors have found that the specific mesoporous silicon dioxide contemplated by the present disclosure can facilitate the controlled release of the API at a generally constant rate over a time period of up to about one day (24 hours) without an excessively depleting or immediate release delivery profile. Such a silicon dioxide is commercially available under the name Parteck® SLP from Millipore/Sigma. Other commercially available mesoporous silicon dioxides that can be used include Syloid®72 FP, 74 FP, 244 FP, and 266 FP from Grace/Mutchler, as well as Sipernat® silica from Evonik. In addition, it should be understood that colloidal/gel, or fumed silicon dioxides are also contemplated by the present disclosure. For example, various Aerosil fumed silicas from Evonik can be utilized, where such fumed silicas can have a surface area ranging from about 75 m²/g to about 500 m²/g, such as from about 100 m²/g to about 475 m²/g, such as from about 125 m²/g to about 450 m²/g, and any ranges therebetween.

Alternatively, the API can be loaded into a carrier powder that is polyvinylpyrrolidone (PVP), such as uncrosslinked PVP. Without intending to be limited by any particular theory, the present inventors have found that the uncrosslinked PVP may function in a polar aprotic nature by structure, the polymer contains a 5-member ring with a tertiary amine and a ketone in a specific arrangement. Thereby, this type of polymer avoids the use of an alcohol (—OH) group excipient, yet provides for a structure which is polar aprotic in nature. Suitable soluble grades of PVP as provided by BASF can includes Kollidon® grades K-12 (molecular weight range 2,000-3,000; pH 4.63), K-17 (molecular weight 7,000-11,000; pH 4.64), K-25 (molecular weight 28,000-34,000; pH 4.00), K-30 (molecular weight 44,000-54,000; pH 4.10), and K-90 (molecular weight 1,000,000-1,500,000; pH 5.68. Other functional polymers may include Kollidon® VA64 (molecular weight range 45,000-70,000, pH 4.51) or other povidones and copolymers thereof by different vendors. Still other uncrosslinked PVPs contemplated by the present disclosure are Plasdone® PVPs available from Ashland, which are copolymers produced by the free radical polymerization of N-vinyl-2-pyrrolidone and vinyl acetate. Such “copovidones” have a molecular weight ranging from about 34,000 to about 43,000. The present inventors have found that the use of polyvinylpyrrolidone in the presence of an API can increase the solubility and stability of the API when used as a carrier powder for introducing the API to other formulation components and for maintaining the API in a solid state.

In some embodiments, the API can be loaded into the PVP at a weight ratio of the API to the PVP ranging from 1:0.5 to about 1:2, such as from about 1:0.6 to about 1:1.8, such as from about 1:0.8 to about 1:1.6, such as at a ratio of about 1:1, and any ranges therebetween. In particular, when the PVP is utilized as the carrier powder for the API, the API can be in amorphous form (e.g., lenalidomide in an amorphous form).

In addition, it is to be understood that the API can first be combined with PVP as a first carrier powder, which can then be added to a second carrier powder that is silicone dioxide. For instance, a blend of 25% to 75% of the API and 75% to 25% of the PVP can be formed, and this resulting powder can be combined blended with silicon dioxide in the ratios discussed above. As a non-limiting example, a blend of 50% LLD and 50% PVP can then be combined with silicone dioxide at a 1:9 ratio of the LLD/PVP blend to the silicon dioxide.

Further, regardless of the particular filler utilized, the amount of carrier powder contained in the formulations contemplated by the present disclosure can range from about 0.5 wt. % to about 15 wt. %, such as from about 1 wt. % to about 12.5 wt. %, such as from about 7 wt. % to about 10 wt. %, or any ranges therebetween, based on the total weight of the formulation for oral administration.

It should be understood that this use of PVP is separate from the use of PVP described below as a controlled release polymer in the formulation. In some embodiments, the PVP can be utilized in conjunction with amorphous lenalidomide, although it is to be understood that the present disclosure also contemplates the use of PVP with any other immunomodulatory imide compound, steroid, or API of interest.

### Filler/Compressing Agents

The formulation can also include one or more fillers or compressing agents. Fillers suitable for use in the formulations of the present disclosure can include lactose, lactose anhydrous, sucrose, starch, pregelatinized starch, dextrose, mannitol (including mannitol Pearlitol SD 200), fructose, xylitol, sorbitol, corn starch, modified corn starch, inorganic salts such as calcium carbonate, calcium phosphate, dicalcium phosphate, calcium sulfate, dextrin/dextrates, maltodextrin, compressible sugars, cellulose derivatives, such as microcrystalline cellulose or wood cellulose (including microcrystalline cellulose 302), or a combination thereof. Examples of lactose that can be used in the formulations of the present disclosure can include, for example, anhydrous lactose, lactose monohydrate, lactose fast flow, directly compressible anhydrous lactose, modified lactose monohydrate, or a combination thereof. In one particular embodiment, the lactose can be a milled lactose monohydrate, such as Pharmatose® 200 M. The lactose can have a build density ranging from about 500 g/L to about 700 g/L, such as from about 525 g/L to about 650 g/L, such as from about 550 g/L to about 600 g/L and/or a median particle size distribution ranging from about 15 μm to about 100 μm, such as from about 20 μm to about 80 μm, such as from about 30 μm to about 60 μm. Examples of microcrystalline cellulose that can be used in the formulations of the present disclosure can include, for example, microcrystalline cellulose such as Avicel® types: PH101, PH102, PH103, PH105, PH 112, PH113, PH200, PH301, other types of microcrystalline cellulose, such as silicified microcrystalline cellulose, or a combination thereof. Other suitable microcrystalline cellulose is commercially available as Microcel® or Tabulose®, a colloidal microcrystalline cellulose, both commercially available from Roquette.

Without intending to be limited by any particular theory, the present inventors have found that the filler, such as lactose, a water soluble component which quickly dissolves (e.g., in less than 10 minutes like an immediate release tablet), creates channels within the tablet to allow even hydration of the polymeric components, which promotes slow, controlled API release in a very uniform manner without creating a hard plug center which never erodes or releases. Thus, up to about 100% API release is obtainable at about 24 hours.

It should be noted that FDA IIG lists lactose as being used at levels up to 2500 mg maximum potency per unit dose and maximum daily exposures of up to 8946 mg per day in solid oral dosage forms including tablets and capsules. It should also be noted that FDA IIG lists microcrystalline cellulose as being used at levels up to 789.6 mg maximum potency per unit dose and maximum daily exposures of up to 20,100 mg per day in solid oral dosage forms including tablets and capsules.

The one or more fillers described above can be present in the formulations contemplated by the present disclosure in an amount ranging from about 15 wt. % to about 85 wt. %, such as from about 20 wt. % to about 80 wt. %, such as from about 30 wt. % to about 75 wt. %, or any ranges therebetween, based on the total weight of the formulation for oral administration.

### Controlled Release Polymer System

The formulation can also include a controlled release polymer system that includes one or more polymers in specific combination. For example, the formulation can include polyvinylpyrrolidone (PVP), such as uncrosslinked PVP, a cellulose and/or a cellulose derivative such as methylcellulose, ethyl cellulose, carboxymethyl cellulose, hydroxypropyl cellulose (HPC), hydroxypropyl methylcellulose (HPMC), an acrylate and/or an acrylate derivative, such as a polyacrylic acid polymer, or a combination thereof.

In one particular embodiment, the formulation can include PVP, such as uncrosslinked PVP, as a controlled release polymer. Without intending to be limited by any particular theory, the present inventors have found that the uncrosslinked PVP may function in a polar aprotic nature by structure, the polymer contains a 5-member ring with a tertiary amine and a ketone in a specific arrangement. Thereby, this type of polymer avoids the use of an alcohol (—OH) group excipient, yet provides for a structure which is polar aprotic in nature. Suitable soluble grades of PVP as provided by BASF can includes Kollidon® grades K-12 (molecular weight range 2,000-3,000; pH 4.63), K-17 (molecular weight 7,000-11,000; pH 4.64), K-25 (molecular weight 28,000-34,000; pH 4.00), K-30 (molecular weight 44,000-54,000; pH 4.10), and K-90 (molecular weight 1,000,000-1,500,000; pH 5.68. Other functional polymers may include Kollidon® VA64 (molecular weight range 45,000-70,000, pH 4.51) or other povidones and copolymers thereof by different vendors. Still other uncrosslinked PVPs contemplated by the present disclosure are Plasdone® PVPs available from Ashland, which are copolymers produced by the free radical polymerization of N-vinyl-2-pyrrolidone and vinyl acetate. Such “copovidones” have a molecular weight ranging from about 34,000 to about 43,000.

In another particular embodiment, the formulation can include HPC. The HPC can have a molecular weight ranging from about 40,000 to about 1,150,000. Suitable soluble grades of HPC as provided by Ashland can include Klucel® grades EF (typical molecular weight 80,000), ELF (typical molecular weight 40,000), LF (typical molecular weight 95,000), JF (typical molecular weight 140,000), GF (typical molecular weight 370,000), MF (typical molecular weight 850,000), or HF (typical molecular weight 1,150,000) or other celluloses thereof by different vendors. In one embodiment, the HPC can be Klucel® EF (typical molecular weight 80,000). Without intending to be limited by any particular theory, the present inventors have found that HPCs in this molecular weight range aid in the release of APIs having low solubility in a controlled manner.

In still another embodiment, the formulation can include polyacrylic acid. In one embodiment, the polyacrylic acid can be crosslinked. One example of a suitable crosslinked polyacrylic acid that is contemplated by the present disclosure is a carbomer, which can be crosslinked with allyl sucrose or allyl pentaerythritol. Examples of pharmaceutical grades of carbomers include, but are not limited to Carbopol® 934 NF, Carbopol® 940 NF, Carbopol® 941 NF, Carbopol® 971, Carbopol® 71 G, Carbopol® 974, Carbopol® 980, Carbopol® 981, Carbopol® 1342, and Carbopol® 1382. Other suitable crosslinked polyacrylic acids include Ultrez® 10, as well as Carbopol® ETD 2020, and Pemulen® TR1, which are crosslinked polyacrylic acid copolymers (e.g., crosslinked copolymers of acrylic acid and a C10-30 alkyl acrylate comonomer, which can be crosslinked with allyl pentaerythritol). Regardless of the polyacrylic acid utilized, the polyacrylic acid can have a Brookfield viscosity (viscosity ranging from about 3,000 centipoise (cP) to about 80,000 cP, such as from about 3,500 cP to about 50,000 cP, such as from about 4,000 cP to about 26,500 cP, as measured at 25° C., RVT, 20 rpm, neutralized to pH of 7.3-7.8.

It should be noted that FDA IIG lists povidone (PVP), and specifically Kollidon K-90, as being used at levels up to 240 mg w/w maximum potency per unit dose and maximum daily exposures of up to 2828 mg per day in solid oral dosage forms including tablets and capsules. It should also be noted that FDA IIG lists hydroxypropyl cellulose (HPC), and specifically Klucel EF, as being used at levels up to 241.84 mg maximum potency per unit dose and maximum daily exposures of up to 960 mg per day in solid oral dosage forms including tablets and capsules. Further, it should be noted that FDA IIG lists polyacrylic acid, and specifically Carbopol 981 NF, as being used at levels up to 195 mg w/w maximum potency per unit dose and maximum daily exposures of up to 600 mg per day in solid oral dosage forms including tablets and capsules.

The one or more controlled release polymers described above can each be present in the formulations contemplated by the present disclosure in an amount ranging from about 1 wt. % to about 50 wt. %, such as from about 2.5 wt. % to about 35 wt. %, such as from about 5 wt. % to about 25 wt. %, or any ranges therebetween, based on the total weight of the formulation for oral administration. Further, when more than one controlled release polymer is utilized as part of a controlled release polymer system, the total amount of controlled release polymers present can range from about 10 wt. % to about 70 wt. %, such as from about 15 wt. % to about 60 wt. %, such as from about 20 wt. % to about 50 wt. %, or any ranges therebetween, based on the total weight of the formulation for oral administration. Without intending to be limited by any particular theory, the present inventors have surprisingly found that the controlled release polymers in the polymer system may act individually as immediate release agents, but when utilized in the specific combinations contemplated by the present disclosure, the polymers provide controlled release of the APIs described herein over a period of up to about 24 hours. Meanwhile, when used alone, the polymers may function in acidic media as immediate release polymers whereby the APIs described herein may be quickly released over a period of about 1-2 hours (e.g., for HPC and PVP) or released in only a slightly controlled manner over a period of about 6-12 hours (e.g., for polyacrylic acid).

### Lubricant/Slip Agent

The formulation can also include one or more lubricants or slip agents. The one or more lubricants or slip agents can be a nonionic surfactant. In some embodiments, the nonionic surfactant can be a poloxamer, which is a nonionic block copolymer of polyoxy(ethylene) and polyoxy(propylene). Examples of suitable poloxamers include polyoxythelene 188, polyoxyethlene 237, polyoxyethylene 338, polyoxyethylene 407, or a combination thereof. Although any suitable poloxamer can be utilized, in one particular embodiment, the poloxamer can be polyoxyethylene 407. Such poloxamers can have a thermogelling temperature above about 25° C., such as from about 25° C. to about 37° C. may be used in the invention. These are commercially available from a number of suppliers, such as BASF under the tradename Pluronic®, or under the tradename Kolliphor®, or Croda under the tradename Synperonic®.

It should be noted that FDA IIG lists polyoxyethylene 407 (PEO), and specifically Kolliphor P407 Micro, as being used at levels up to 107 mg maximum potency per unit dose and maximum daily exposures of up to 495 mg per day in solid oral dosage forms including tablets and capsules.

The lubricant or slip agent can be present in the formulations contemplated by the present disclosure in an amount ranging from about 0.5 wt. % to about 15 wt. %, such as from about 1 wt. % to about 12.5 wt. %, such as from about 2.5 wt. % to about 10 wt. %, or any ranges therebetween, based on the total weight of the formulation for oral administration.

Without intending to be limited by any particular theory, the present inventors have found that the lubricant or slip agents described above can be used in combination with the controlled release polymer system and filler contemplated by the present disclosure to facilitate even hydration of a tablet for oral administration, where the specific combination of excipients “swells” the tablet but do not simply disperse or dissolve in the presence of dissolution media. Thus, the characteristics of lactose (immediately solubilizes), HPC (slowly hydrates and dissolves), PVP (slowly hydrates and dissolves) and poloxamer (slowly hydrates and dissolves) control the aspects of how the polyacrylic acid polymer behaves as a very slowly hydrating polymeric system with no total solubilization.

### Additional Excipients

The present disclosure can also include one or more additional excipients. Examples can include disintegrating/dispersing agents, such as crospovidone or croscarmellose sodium; lubricating/glidant agents, such as magnesium stearate; antioxidants, such as BHT or BHA or propyl gallate; preservatives, such as methyl-paraben or propyl-paraben; or other solid oral excipients or grades thereof for which any of these materials are at an acceptable level based on FDA IIG recommended limits in solid oral dosage forms including tablets and capsules.

Such excipients can each be present in the formulations contemplated by the present disclosure in an amount ranging from about 0.05 wt. % to about 10 wt. %, such as from about 0.1 wt. % to about 7.5 wt. %, such as from about 0.5 wt. % to about 5 wt. %, or any ranges therebetween, based on the total weight of the formulation for oral administration.

### Enteric Coating or Enteric Capsule

The formulation can also include an enteric coating or an enteric capsule shell to enhance the controlled release capabilities, particularly to avoid the release of the API in a low pH environment, such as in the stomach. The materials to enable such a coating are known by those of skill in the art and can be present in the formulations contemplated by the present disclosure. The materials of such a coating may include but not limited to cellulose acetate isobutyrate, cellulose acetate phthalate, ethylcellulose, hydroxypropyl cellulose or hydroxypropyl methyl cellulose or hydroxypropyl methyl cellulose phthalate (HPMC-P) by Ashland or Eastman Chemical, Eudragits by Evonik in grades such as L, S, E PO, RS, ES, or FS, Acry-Eze® by Colorcon or combinations thereof. The materials of the coating may be mono-layer or multi-layer in composition to obtain a preferred release in custom pH range from about pH 5.0 up to about pH 7.8 physiological buffer solutions. The materials may be applied in a convection air coating process from water-based or organic solvent-based solutions in an amount ranging from about 1 wt. % to about 30 wt. % gain of the core controlled release matrix tablet weight, such as from about 2 wt. % to about 20 wt. %, such as from about 3 wt. % to about 15 wt. %, or any ranges therebetween, based on the total weight of the formulation for oral administration. The initial coating or tablet seal coating may be applied as an immediate release coating that has a concentration ranging from about 1 wt. % to about 10 wt. %, such as from about 2 wt. % to about 8 wt. %, such as from about 3 wt. % to about 7 wt. %, or any ranges therebetween, based on the total weight of the tablet. Further, it should be understood that the enteric coating may be applied as a single or multiple layer coating with or without the presence of a seal coating that has a concentration ranging from about 1 wt. % to about 30 wt. %, such as from about 2 wt. % to about 20 wt. %, such as from about 3 wt. % to about 15 wt. %, or any ranges therebetween, based on the total weight of the uncoated or seal coated tablet.

Alternatively to an enteric coated tablet, a hydroxypropyl methylcellulose (HPMC)/hydroxypropyl methylcelluse phthalate (HPMC-P) mixture can be utilized to produce enteric capsules which may be filled with the dry powder or granulated controlled release polymer blends. Examples of these enteric capsules include but not limited to Capsuline's Acid Resistant Capsules, Capsugel Enprotect™, Evonik Eudracap®, or Gelita's Enteric Gelatin Capsules. Further it should be understood that the enteric capsule shell is a shell comprised of a monolithic shell or multilayer shell to function as an enteric coating. Enteric capsule shells can include a blend of HPMC/HPMC-P at a ratio of about 35:65 to about 50:50 and can have a weight of about 10% to about 40%, such as about 25%, of the total capsule weight with the filled controlled release formulation.

### Method of Making

In one embodiment, the present disclosure contemplates a method of making a formulation for oral administration. The method includes combining an active pharmaceutical ingredient (API) having a water solubility of less than about 10 mg/mL and a melting point of greater than about 120° C. with a first solvent to form a solution; combining a carrier powder with a second solvent to form a suspension; combining the solution and the suspension to form a blend; drying the blend; forming a dry powder from the blend; and combining the dry powder with a filler and a polymer system comprising one or more controlled release polymers to form a mixture.

The first solvent can include a polar aprotic solvent and the second solvent can include ethyl acetate. The mixture can be compressed into tablet form. Alternatively, the mixture can be introduced into a capsule shell to form a capsule for oral administration.

In a second embodiment, the present disclosure contemplates a method of making a formulation for oral administration. The method includes combining an active pharmaceutical ingredient (API) having a water solubility of less than about 10 mg/mL and a melting point of greater than about 120° C., where the API is prepared separately as an amorphous combination with PVP in about a 1:1 ratio; combining any excipient dry powders; forming a dry powder blend; and combining the resulting dry powder blend with a filler and a polymer system comprising one or more controlled release polymers to form a mixture.

The mixture can then be compressed into tablet form. Alternatively, the mixture can be introduced into a capsule shell to form a capsule for oral administration.

In yet a third embodiment, the present disclosure contemplates a method of making a formulation for oral administration. The method includes combining an active pharmaceutical ingredient (API) having a water solubility of less than about 10 mg/mL and a melting point of greater than about 120° C. with dry powder addition; combining any excipient dry powders; forming a dry powder blend; and combining the resulting dry powder blend with a filler and a polymer system comprising one or more controlled release polymers to form a mixture.

The mixture can then be compressed into tablet form. Alternatively, the mixture can be introduced into a capsule shell to form a capsule for oral administration.

### API Delivery

The present disclosure contemplates that formulations of APIs having low water solubility (e.g., less than about 10 mg/mL) and high melting points (greater than about 120° C.), with short half-lives, can be delivered through a tablet or capsule in oral form with desirable release kinetics to support a near zero order delivery profile and in a controlled release form, as shown by the HPLC analysis and release profiles below, which are compared to the exiting immediate release formulations available commercially.

The present disclosure is further described in the following Examples, which do not limit the scope of the disclosure described in the claims.

### Example 1

The first conceptual formulation evaluated a 1 wt. % lenalidomide loading with 9 wt. % Parteck SLC, a mesoporous silicon dioxide, as a premixed, solvated mixture which was then tray dried and sieved to <75 μm. This mixture was then added to 10 wt. % HPC as Klucel® EF, 10 wt. % PVP as Kollidon® K-90, 10 wt. % Carbopol® 981 NF (a crosslinked polyacrylic acid), and qs to 100 wt. % with milled lactose (e.g., 60 wt. %). Tablets were compressed manually with a 10 mm round tablet tooling and a 2 lb hammer. Tablets were difficult to release from the tool die, warranting the investigation of the addition of slip agents or glidants into new formulations. Each tablet weighed to 500 mg+/−25 mg.

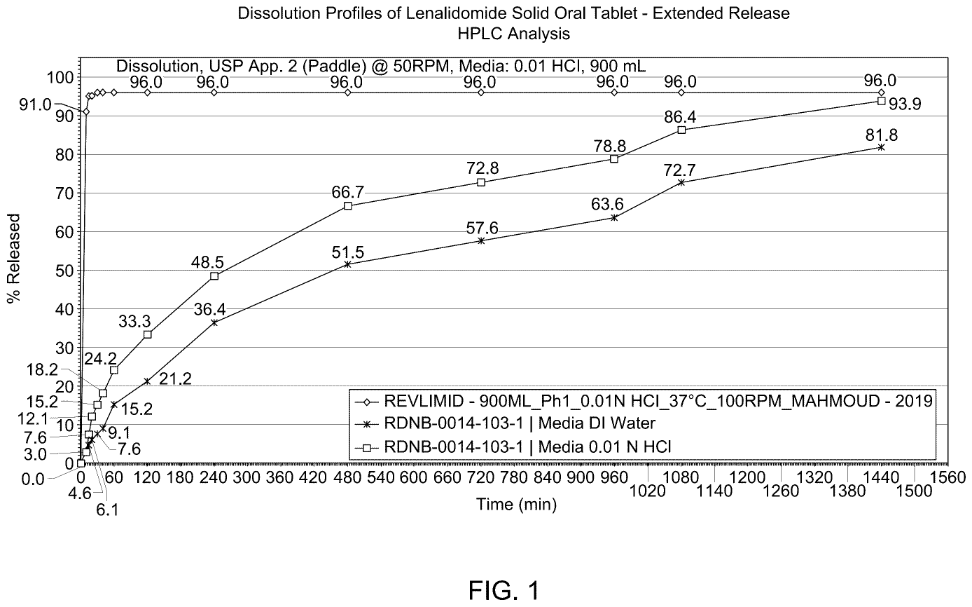

Thereafter, and referring to FIG. 1 and Table 5 below, it was determined that the combination of polymers and excipients described above in conjunction with lenalidomide was able to achieve a uniform and consistent release profile of lenalidomide in two (2) different pH environments, including DI Water (relatively neutral) and 0.01 N HCl (gastric acid). The dissolution profiles were compared to immediate release lenalidomide (e.g., Revlimid®) as described in the article by Mahmoud (2019) referenced above.

TABLE 5
Dissolution Results for Formulation RDNB-0014-103-1 in DI Water and 0.01N HCl dissolution media

| | Time (minutes) and Respective % of API Released | | | | | | | | | | | | | |
|---|---|---|---|---|---|---|---|---|---|---|---|---|---|---|
| Formulation | 0 | 10 | 15 | 20 | 30 | 40 | 60 | 120 | 240 | 480 | 720 | 960 | 1080 | 1440 |
| Revlimid | 0.0 | 91.0 | 95.0 | 95.0 | 96.0 | 96.0 | 96.0 | 96.0 | 96.0 | 96.0 | 96.0 | 96.0 | 96.0 | 96.0 |
| 900 ML Ph 1 | | | | | | | | | | | | | | |
| 0.01N HCl | | | | | | | | | | | | | | |
| 37° C. 100 RPM | | | | | | | | | | | | | | |
| Mahmoud | | | | | | | | | | | | | | |
| (2019) | | | | | | | | | | | | | | |
| RDNB-0014- | 0.0 | 3.0 | 4.6 | 6.1 | 7.6 | 9.1 | 15.2 | 21.2 | 36.4 | 51.5 | 57.6 | 63.6 | 72.7 | 81.8 |
| 103-1 | | | | | | | | | | | | | | |
| DI Water | | | | | | | | | | | | | | |
| RDNB-0014- | 0.0 | 3.0 | 7.6 | 12.1 | 15.2 | 18.2 | 24.2 | 33.3 | 48.5 | 66.7 | 72.8 | 78.8 | 86.4 | 93.9 |
| 103 -1 | | | | | | | | | | | | | | |
| 0.01N HCl | | | | | | | | | | | | | | |

As shown by FIG. 1 and Table 5, the formulations contemplated by the present disclosure show a gradual, controlled release of lenalidomide over 24 hours (1440 minutes), where only 3% of the API was released within 10 minutes in 0.01 N HCl, with a slow release of levels between 81.8% and 93.9% after 24 hours, as compared to the immediate release lenalidomide (Revlimid®), where 91% of the API was released within 10 minutes in 0.01N HCl and plateaued at 96% after just 30 minutes.

### Example 2

Next, various formulations contemplated by the present disclosure were then tested to better understand the performance of variations in the excipient loading to evaluate drug release. The formulations listed below evaluated +/−50% of polymer loading in which all three control release polymers (HPC, PVP, and carbomer) were reduced or increased by 50% from the target of 10 wt. % loading. The dissolution profiles were compared to immediate release lenalidomide (e.g., Revlimid®) as described in the article by Mahmoud (2019) referenced above.

TABLE 6
Example 2 Formulation Components by Wt. %

| | RDNB-0014- | RDNB-0014- | RDNB-0014- |
|---|---|---|---|
| | 104-1 | 104-2 | 104-3 |
| | (Target | (Decrease 50% | (Increase 50% |
| Ingredient | Formula) | Polymers) | Polymers) |
| 10% Lenalidomide HH | 10% (1% LLD) | 10% (1% LLD) | 10% (1% LLD) |
| in SiO₂ | | | |
| Klucel EF (HPC) | 10% | 5% | 15% |
| Kollidon K-90 (PVP) | 10% | 5% | 15% |
| Carbopol 981 NF | 10% | 5% | 15% |
| (Carbomer | | | |
| Homopolymer | | | |
| Type A USP) | | | |
| Lactose | 60% | 75% | 45% |

TABLE 7
Dissolution Results for Formulation RDNB-0014-104-1 to 3

| | Time (minutes) and Respective % of API Released | | | | | | | | | | | | | | |
|---|---|---|---|---|---|---|---|---|---|---|---|---|---|---|---|
| Formulation | 0 | 10 | 15 | 20 | 30 | 40 | 45 | 60 | 120 | 240 | 480 | 720 | 960 | 1080 | 1440 |
| Revlimid | 0.0 | 91.0 | 95.0 | 95.0 | 96.0 | 96.0 | 96.0 | 96.0 | 96.0 | 96.0 | 96.0 | 96.0 | 96.0 | 96.0 | 96.0 |
| 900 ML Ph 1 | | | | | | | | | | | | | | | |
| 0.01N HCl | | | | | | | | | | | | | | | |
| 37° C. 100 RPM | | | | | | | | | | | | | | | |
| Mahmoud | | | | | | | | | | | | | | | |
| (2019) | | | | | | | | | | | | | | | |
| RDNB-0014- | 0.0 | 7.0 | 10.1 | 13.1 | 16.2 | 19.2 | 20.8 | 25.6 | 35.5 | 50.6 | 67.1 | 78.8 | 86.5 | 90.3 | 90.3 |
| 104-1 | | | | | | | | | | | | | | | |
| RDNB-0014- | 0.0 | 9.6 | 12.3 | 14.9 | 18.6 | 22.2 | 23.9 | 28.9 | 40.2 | 56.0 | 74.2 | 85.7 | 90.1 | 92.3 | 92.3 |
| 104-2 | | | | | | | | | | | | | | | |
| RDNB-0014- | 0.0 | 6.1 | 8.8 | 11.4 | 14.3 | 17.1 | 18.3 | 21.8 | 30.3 | 43.3 | 58.3 | 72.6 | 82.2 | 87.0 | 87.0 |
| 104-3 | | | | | | | | | | | | | | | |

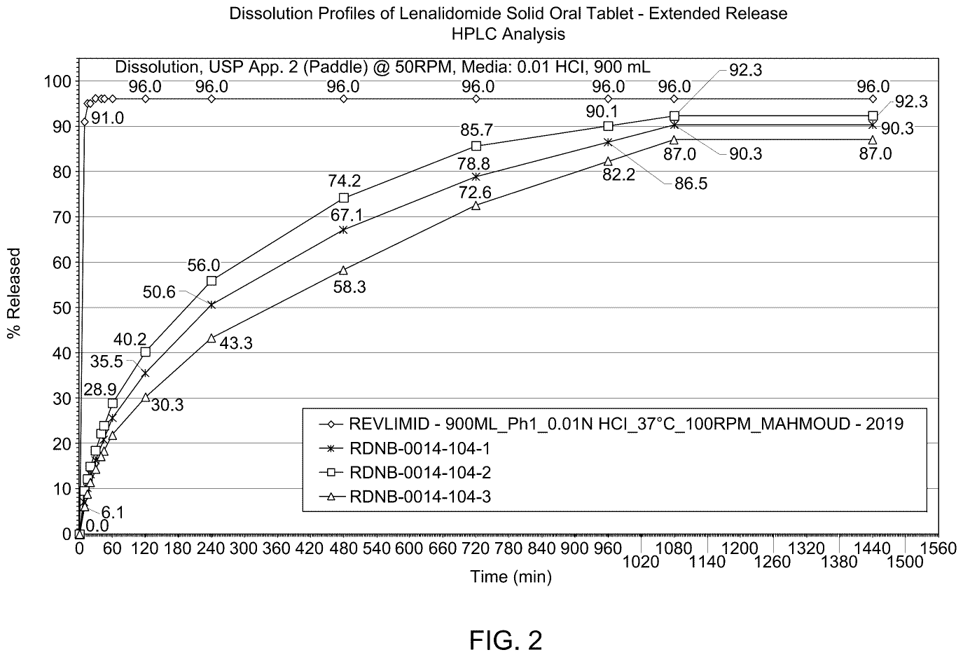

As shown by FIG. 2 and Table 7, the formulations contemplated by the present disclosure show a gradual, controlled release of lenalidomide over 24 hours (1440 minutes), where only 6.1%-7.0% of the API was released within 10 minutes in 0.01N HCl, with a slow release of levels between 87.0% and 92.3% after 24 hours, as compared to the immediate release lenalidomide (Revlimid®), where 91% of the API was released within 10 minutes in 0.01 N HCl and plateaued at 96% after just 30 minutes.

### Example 3

Next, various formulations contemplated by the present disclosure were made to support the range of polymers used to elicit the controlled release effect of the formulation with a range of HPC (5 wt. % to 15 wt. %), PVP (5 wt. % to 15 wt. %), and Carbomer (5 wt. % to 15 wt. %), where the weight percentage of each was set at 10 wt. %. A combination of these three (3) polymers is necessary to achieve controlled release. Lactose is a filler and compressing agent to make the tablet. Additionally, poloxamer P407 is added to the formulation at 5% to support the controlled release nature of the formulation, as a slip agent to reduce friction during tablet manufacturing and is anticipated to have an impact on improved solubility in the intestinal tract. Further, in this Example, the use of lenalidomide hemihydrate in silicon dioxide carrier powder is compared to the use of amorphous lenalidomide in PVP carrier powder to determine the effect, if any, on the release of the lenalidomide.

TABLE 8
Example 3 Formulation Components by Wt. %

| | RDNB-0014- | RDNB-0014- | RDNB-0014- |
|---|---|---|---|
| Ingredient | 104-4 | 105-9 | 105-10 |
| 10% Lenalidomide HH | 20% (2% LLD) | 0% | 10% (1% LLD) |
| in SiO₂ | | | |
| 50% LLD:50% PVP | 0% | 2% (1% LLD) | 0% |
| Silicon Dioxide | 18% (as LLD | 8% | 9% (as LLD |
| (Parteck SLC) | blend above) | | blend above) |
| Klucel EF (HPC) | 10% | 10% | 10% |
| Kollidon K-90 (PVP) | 10% | 10% | 10% |
| Carbopol 981 NF | 10% | 10% | 10% |
| (Carbomer | | | |
| Homopolymer | | | |
| Type A USP) | | | |
| Kolliphor P407 Micro | 0% | 0% | 5% |
| (Poloxamer 407) | | | |
| Lactose | 50% | 60% | 55% |

TABLE 9
Dissolution Results for Formulations RDNB-0014-104-4, 105-9, and 105-10

| | Time (minutes) and Respective % of API Released | | | | | | | | | | | | | | |
|---|---|---|---|---|---|---|---|---|---|---|---|---|---|---|---|
| Formulation | 0 | 10 | 15 | 20 | 30 | 40 | 45 | 60 | 120 | 240 | 480 | 720 | 960 | 1080 | 1440 |
| Revlimid | 0.0 | 91.0 | 95.0 | 95.0 | 96.0 | 96.0 | 96.0 | 96.0 | 96.0 | 96.0 | 96.0 | 96.0 | 96.0 | 96.0 | 96.0 |
| 900 ML Ph 1 | | | | | | | | | | | | | | | |
| 0.01N HCl | | | | | | | | | | | | | | | |
| 37° C. 100 RPM | | | | | | | | | | | | | | | |
| Mahmoud | | | | | | | | | | | | | | | |
| (2019) | | | | | | | | | | | | | | | |
| RDNB-0014- | 0.0 | 7.1 | 9.3 | 11.5 | 15.1 | 18.6 | 20.0 | 24.3 | 32.0 | 44.7 | 59.7 | 74.0 | 82.2 | 86.3 | 96.3 |
| 104-4 | | | | | | | | | | | | | | | |
| RDNB-0014- | 0.0 | 7.6 | 10.4 | 13.2 | 16.8 | 20.3 | 21.9 | 26.5 | 37.9 | 49.5 | 66.5 | 79.0 | 86.5 | 90.3 | 90.3 |
| 105-9 | | | | | | | | | | | | | | | |
| RDNB-0014- | 0.0 | 7.3 | 9.9 | 12.5 | 16.8 | 21.0 | 22.2 | 25.8 | 34.2 | 47.4 | 65.2 | 76.2 | 84.7 | 88.9 | 88.9 |
| 105-10 | | | | | | | | | | | | | | | |

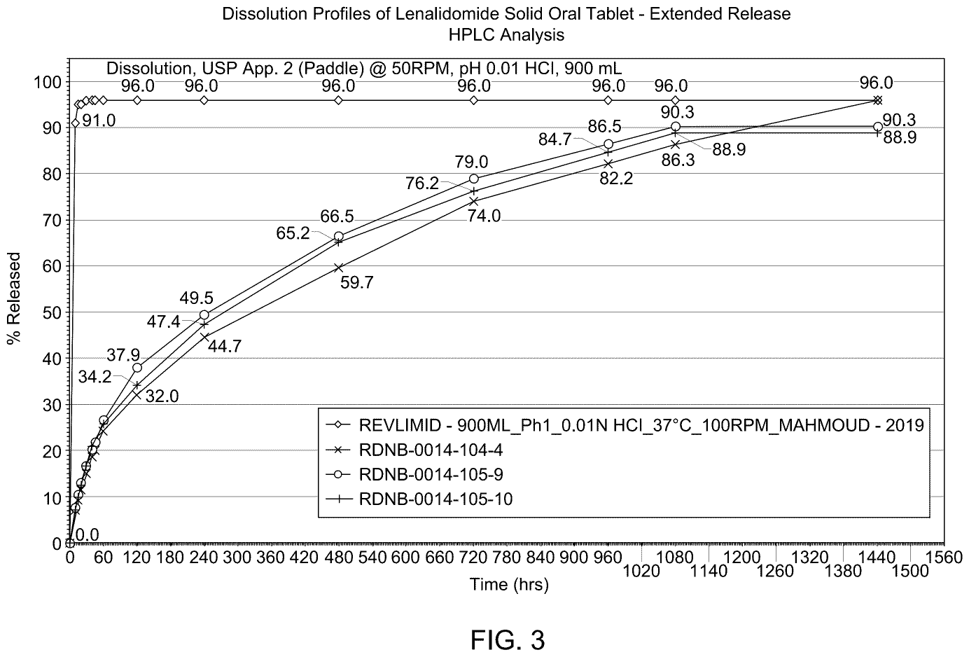

As shown by FIG. 3 and Table 9, the formulations contemplated by the present disclosure show a gradual, controlled release of lenalidomide over 24 hours (1440 minutes), where only 7.1%-7.3% of the API was released within 10 minutes in 0.01N HCl, with a slow release of levels between 88.9% and 96.3% after 24 hours, as compared to the immediate release lenalidomide (Revlimid®), where 91% of the API was released within 10 minutes in 0.01 N HCl and plateaued at 96% after just 30 minutes. Further, this Example shows that the formulations of the present disclosure support the use of both silicon dioxide and PVP as a carrier powder for the lenalidomide for controlled release and that the addition of a poloxamer can support such controlled release.

### Example 4

In Example 4, various formulations contemplated by the present disclosure were formed to explore the effect of lenalidomide in combination with each of HPC, K-90 and Carbopol 981 individually and in combination as dry powder blends with direct tablet compression.

TABLE 10
Example 4 Formulation Components by Wt. %

| | RDNB- | RDNB- | RDNB- | RDNB- |
|---|---|---|---|---|
| | 0014- | 0014- | 0014- | 0014- |
| Ingredient | 106-4 | 107-6 | 107-7 | 107-8 |
| Lenalidomide Hemihydrate | 2% | 2% | 2% | 2% |
| Silicon Dioxide (Parteck | 9% | 9% | 9% | 9% |
| SLC) | | | | |
| Kollidon K-90 (PVP) | 10% | 10% | 0% | 0% |
| Carbopol 981 NF | 10% | 0% | 10% | 0% |
| (Carbomer Homopolymer | | | | |
| Type A USP) | | | | |
| Klucel EF (HPC) | 10% | 0% | 0% | 10% |
| Kolliphor P407 Micro | 5% | 5% | 5% | 5% |
| (Poloxamer 407) | | | | |
| Lactose | 54% | 74% | 74% | 74% |

TABLE 11
Dissolution Results for Formulations RDNB-0014-106-4, 107-6, 107-7, and 107-8

| | Time (minutes) and Respective % of API Released | | | | | | | | | | | | | | |
|---|---|---|---|---|---|---|---|---|---|---|---|---|---|---|---|
| Formulation | 0 | 10 | 15 | 20 | 30 | 40 | 45 | 60 | 120 | 240 | 360 | 720 | 960 | 1080 | 1440 |
| Revlimid | 0.0 | 91.0 | 95.0 | 95.0 | 96.0 | 96.0 | 96.0 | 96.0 | 96.0 | 96.0 | 96.0 | 96.0 | 96.0 | 96.0 | 96.0 |
| 900 ML Ph 1 | | | | | | | | | | | | | | | |
| 0.01N HCl | | | | | | | | | | | | | | | |
| 37° C. 100 RPM | | | | | | | | | | | | | | | |
| Mahmoud | | | | | | | | | | | | | | | |
| (2019) | | | | | | | | | | | | | | | |
| RDNB-0014- | 0.0 | 2.3 | 3.5 | 4.5 | 6.6 | 8.3 | 9.2 | 11.8 | 20.0 | 31.0 | 40.8 | 58.5 | 67.0 | 71.2 | 82.8 |
| 106-4 | | | | | | | | | | | | | | | |
| RDNB-0014- | 0.0 | 20.9 | 31.3 | 40.4 | 58.6 | 65.4 | 68.9 | 79.1 | 87.9 | 92.6 | 95.4 | 99.4 | 98.0 | 97.3 | 96.4 |
| 107-6 | | | | | | | | | | | | | | | |
| RDNB-0014- | 0.0 | 4.1 | 6.1 | 7.6 | 10.7 | 13.6 | 15.0 | 19.3 | 34.4 | 56.9 | 74.9 | 98.1 | 105.2 | 108.7 | 111.4 |
| 107-7 | | | | | | | | | | | | | | | |
| RDNB-0014- | 0.0 | 33.7 | 50.5 | 56.6 | 68.9 | 72.6 | 74.4 | 79.9 | 90.0 | 97.8 | 101.9 | 105.2 | 106.3 | 106.8 | 107.9 |
| 107-8 | | | | | | | | | | | | | | | |

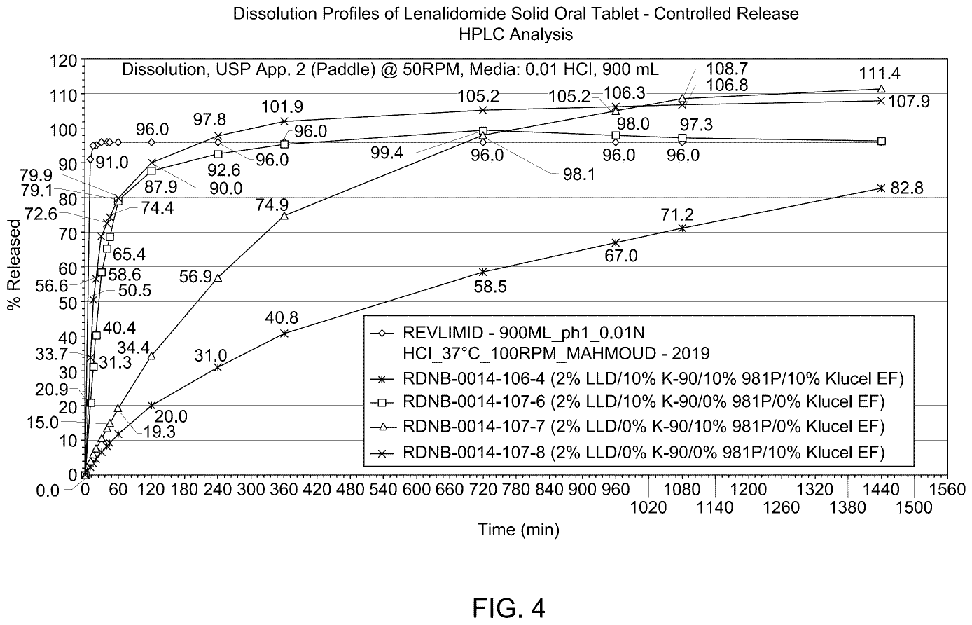

The results in Table 11 and FIG. 4 show that the release profile is significantly controlled based on release rate over 24 hours in which the formulations with the individual polymers, K-90 (107-6) and HPC (107-8), are very close to an immediate release dosage form and where Carbopol 981 NF alone (107-7) is between the preferred formulation and the release rate of the immediate release, Revlimid®. The formulation containing all three polymers (106-4) provides the most gradual, controlled release of lenalidomide. For example, it showed a gradual, controlled release of lenalidomide over 24 hours (1440 minutes), where only 2.3% of the API was released within 10 minutes in 0.01 N HCl, with a slow release of 82.8% after 24 hours, as compared to the immediate release lenalidomide (Revlimid®), where 91% of the API was released within 10 minutes in 0.01N HCl and plateaued at 96% after just 30 minutes. Further, the 107-6 and 107-formulations with only one polymer showed release rates of 79.1% and 79.9%, respectively, after just 1 hour.

Of note is that Klucel® EF is actually a marketed hydroxypropyl cellulose (HPC) polymer for immediate release dosage forms, in the range of 2 wt. %-6 wt. %, and the higher molecular weights are intended for controlled release at levels above 15 wt. %. As shown, a concentration of 10 wt. % Klucel® EF alone would support an immediate release dosage form, but the release rate would not be obvious to support a controlled release profile where the tablet is completely dissolved in the dissolution media within about 1 hour.

Similarly, the use of PVP, as Kollidon® 90, a high molecular weight povidone (PVP), is marketed as a controlled release polymer. It was found in this study that a concentration of 10 wt. % Kollidon® 90 alone would support an immediate release dosage form, but the release rate would not be obvious to support a controlled release profile where the tablet is completely dissolved in the dissolution media within about 1 hour.

Meanwhile, Carbopol® 981P provides a controlled delivery in the present composition with a 10 wt. % loading in the tablet. The profile would support a controlled release through about 6 to about 8 hours.

Surprisingly, it was found that the combination of these polymers actually act in unison to provide sufficient wetting pathways to uniformly hydrate the polymeric composition of Carbomer, PVP, and hydroxypropyl cellulose and provide for a most preferred near-zero order continuous release of lenalidomide over about 24 hours. One the other hand, PVP and HPC do not act as would be expected in the immediate release profiles observed and instead, with the Carbopol® polymer, offer a controlled rate of hydration for the Carbopol® polymer and impart a preferred consistent release rate from time point to time point promoting a true near-zero release profile.

This Example shows that the combination of all three polymers with lactose is expected to create a uniform structure of a water hydration event present within the swellable tablet, and such that the lactose is completely water soluble and offers a preferred controlled release rate where the channels formed within the tablet upon hydration allow a completely controlled release without possibility of dose dumping.

### Example 5

In Example 5 and referring to Table 12, the basic compositions for lenalidomide that were produced successfully and within current FDA inactive ingredient database guidelines are shown, with the understanding that other ranges are contemplated by the present disclosure.

TABLE 12
Proposed Formulations of LLD Systems

| | Generic | % | Amount (mg/ |
|---|---|---|---|
| Ingredients | Name | Composition | tablet) |
| Lenalidomide | Lenalidomide | 0.2%-5% | 1 mg to 25 mg |
| Kollidon 90 | Povidone | 5%-15% | 25 mg to |
| | (PVP) | | 75 mg |
| Klucel EF | Hydroxypropyl | 5%-15% | 25 mg to |
| | cellulose | | 75 mg |
| | (HPC) | | |
| Carbopol 981 | Carbomer | 0.5%-15% | 25 mg to |
| | Homopolymer | | 75 mg |
| | Type A | | |
| Lactose | Lactose | Qs: 30%-86% | 150 mg to |
| Parteck SLC | Silicon dioxide | 1%-10% | 410 mg |
| | (SiO2) | | 5 mg to 50 mg |
| Kolliphor P407 | Poloxamer 407 | 1%-10% | 5 mg to 50 mg |

| | | |
|---|---|---|
| Target Tablet or Capsule | 500 mg | 500 mg |
| Fill Weight | | |

The API can be added into the dry powder blending process as one of the following:
1) Pure Lenalidomide Hemihydrate D90: 104 to 187 μm
2) Pure Lenalidomide
3) Lenalidomide Hemihydrate dissolved in NMP added to a solvated Parteck SLC (silicon dioxide) in ethyl acetate to create a solubilized suspension of drug and silicon dioxide. The resulting suspended particles were tray dried to dryness and achieved ICH solvent levels for NMP and ethyl acetate. The dried powder is then sieved through a 75 μm mesh to a uniform particle size of <75 μm. Loading was between 10% LLD to 30% LLD in the presence of silicon dioxide.
4) Lenalidomide prepared as a 1:1 ratio of Lenalidomide to povidone in an amorphous material as is commercially available from Dr. Reddy's Laboratory.

Regardless of the state of the API as pure, crystalline, partially crystalline (blend with SiO₂ item 3 above), hydrate, anhydrous, or amorphous (blend with PVP, item 4 above).

It is contemplated that the composition of Lenalidomide with HPC, PVP, Carbomer, Poloxamer 407, silicon dioxide and lactose will achieve:
1) A reduction in drug loading that is feasible to achieve an equivalent AUC with reduced Cmax with a controlled steady and predictable release of drug from the tablet/capsule compared to the Revlimid® based on in silico modeling.
2) A reduction in Cmax by about 50% from about 413 ng/mL from Revlimid 25 mg to about 200 ng/mL from controlled release dosage form.
3) An increase in Tmax from <1 hour with Revlimid® to about 6 hours-12 hours.

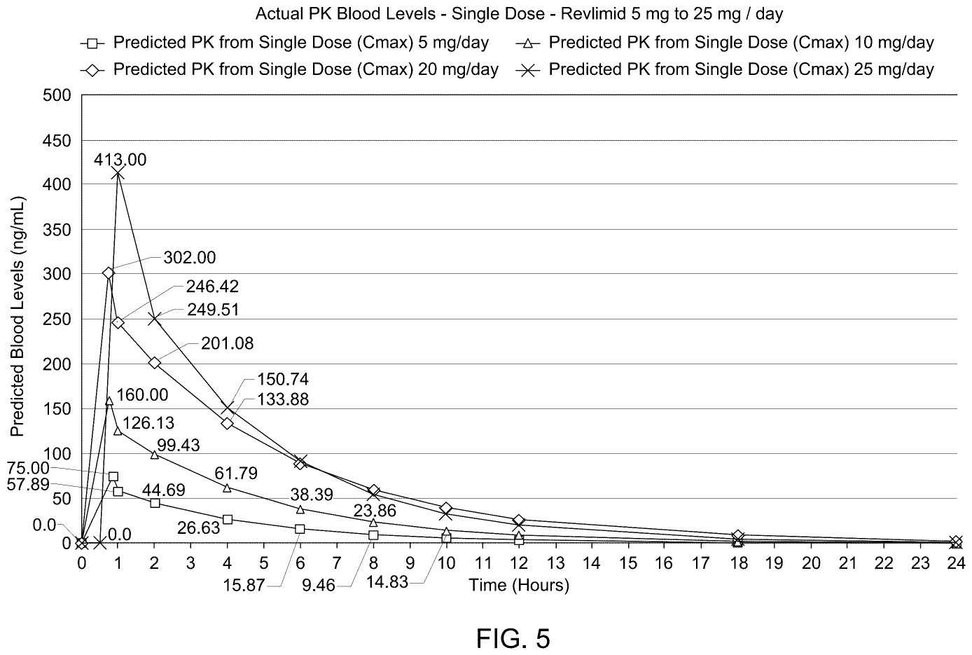

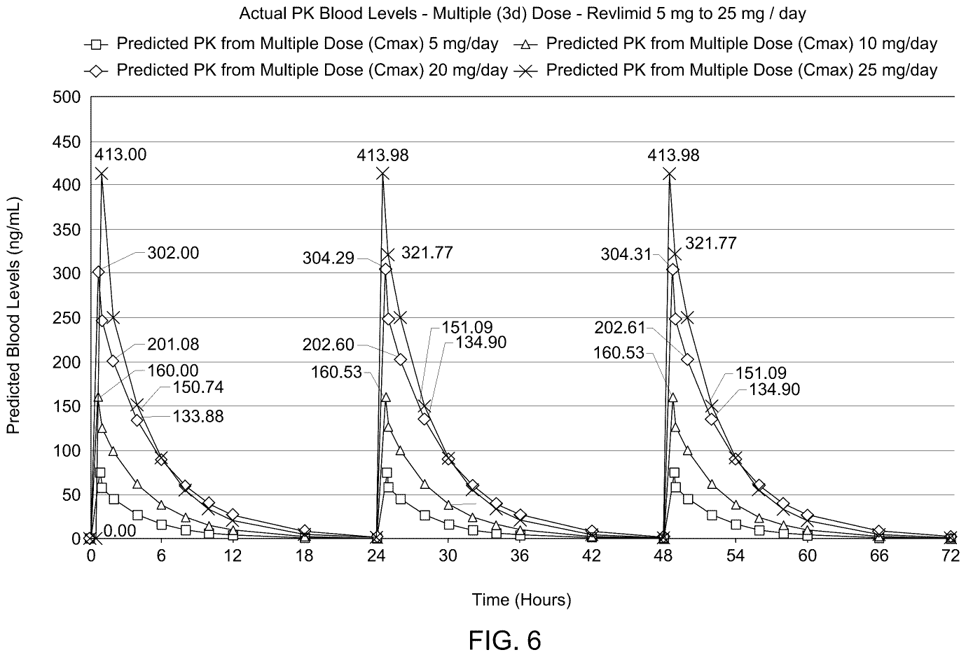

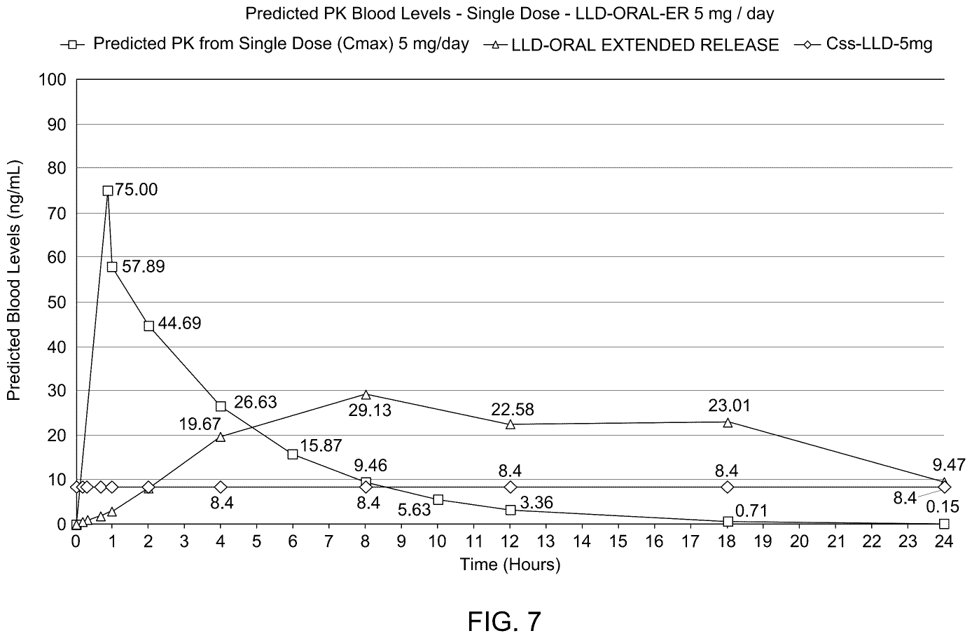

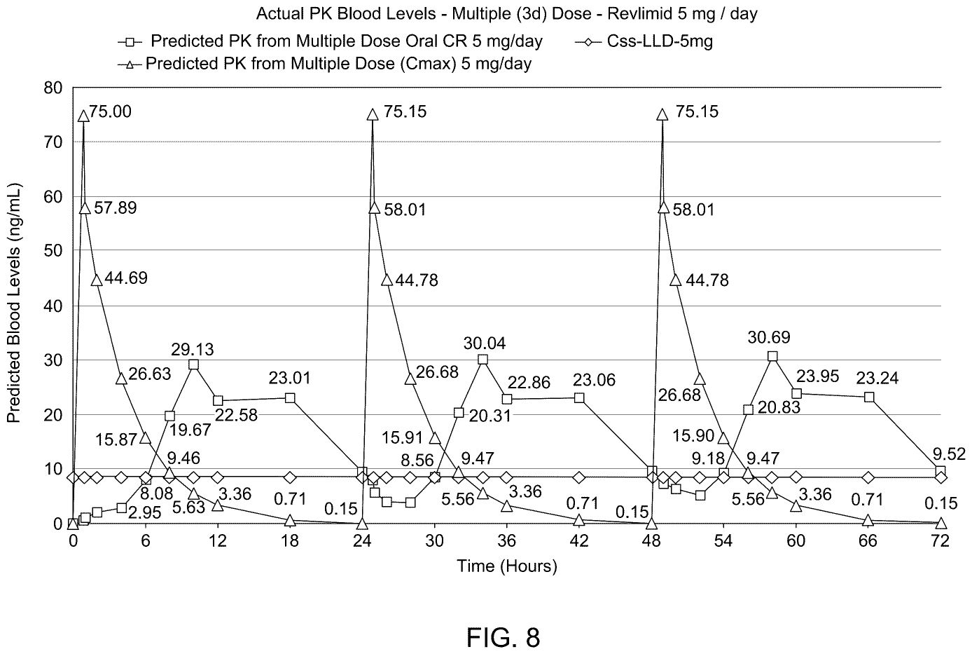

Further, referring to FIGS. 5 and 6, the PK blood levels for single and multiple day dosing of Revlimid® illustrate a typical dump-and-die profile typically associated with the oral delivery of short half-life APIs, an issue that the present disclosure solves. In particular, FIGS. 7 and 8 illustrate the PK blood levels of the controlled release solid oral dosage forms contemplated by the present disclosure compared to the immediate release dosage form with a target blood level calculated at 24 hours of about 8.4 ng/mL based on earlier work from the 5 mg dosage form, suggesting the 5 mg equivalent controlled release profile may be reduced by about 60%, such that about a 2 mg oral dose with the same profile would be in line with the target blood level concentration. In addition, the controlled release solid oral dosage forms contemplated by the present disclosure are compared to Cₛₛ levels, which are the subcutaneous formulation blood levels targeted to achieve 100 mcg/hour to 600 mcg/hour delivery rates. Other doses were modeled and are proportional based on % release IVRT dissolution profile.

### Example 6

Next, in Example 6, various formulations contemplated by the present disclosure were formed to explore the effect of dexamethasone in combination with HPC, K-90 and Carbopol 981, as shown below in Table 13.

TABLE 13
Example 6 Formulation Components by Wt. %

| Ingredient | RDNB-0014-108-1 | RDNB-0014-108-3 |
|---|---|---|
| Dexamethasone | 1% | 1% |
| Silicon Dioxide (Parteck SLC) | 9% | 9% |
| Kollidon K-90 (PVP) | 5% | 15% |
| Carbopol 981 NF (Carbomer | 5% | 15% |
| Homopolymer Type A USP) | | |
| Klucel EF (HPC) | 5% | 15% |
| Kolliphor P407 Micro | 5% | 5% |
| (Poloxamer 407) | | |
| Lactose | 70% | 40% |

TABLE 14
Dissolution Results for Formulations RDNB-0014-108-1 and 108-3

| | Time (minutes) and Respective % of API Released | | | | | | | | | |
|---|---|---|---|---|---|---|---|---|---|---|
| Formulation | 0 | 15 | 30 | 60 | 120 | 240 | 360 | 720 | 1080 | 1440 |
| HEMADY - Std IR release | 0.0 | 40.0 | 80.0 | 90.0 | 95.0 | 95.0 | 95.0 | 95.0 | 95.0 | 95.0 |
| NLT 80% @ 30 min | | | | | | | | | | |
| RDNB-0014-108-1 | 0.0 | 2.2 | 4.0 | 6.7 | 11.0 | 16.4 | 20.9 | 29.8 | 37.8 | 44.7 |
| RDNB-0014-108-3 | 0.0 | 1.3 | 2.6 | 4.4 | 7.7 | 12.8 | 17.3 | 26.4 | 34.7 | 41.5 |

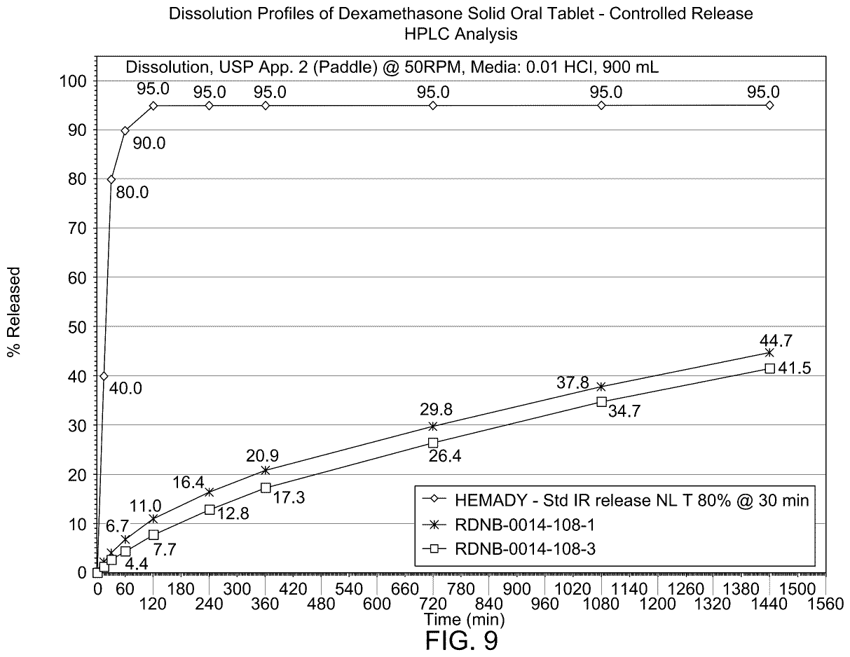

As shown in Table 14 and FIG. 9, and based on the knowledge gleaned from the reference dissolution from lenalidomide highlighting the differences in delivery of HPC vs. povidone vs. carbomer, it became clear that manipulation of the polymer concentrations, specifically the carbomer concentration, within these formulations will have a significant impact on the release rate of drug from these swellable tablets. The initial concept supported above is in a limited range around 10% (±5%), and the amount of HPC and povidone as a function of swellability to control the rate of hydration of the carbomer is expected to further increase the release rate with lower amounts of Carbopol while keeping HPC and povidone at target concentrations with specific drugs, such as dexamethasone, to improve the release rate from the graph above to achieve NLT 80% release at 24 hours. As compared to the immediate release HEMADY® formulation,

Further, the formulations contemplated by the present disclosure show a gradual, controlled release of dexamethasone over 24 hours (1440 minutes), where only 1.3%-2.2% of the API was released within 15 minutes in 0.01 N HCl, with a slow release of levels between 88.9% and 96.3% after 24 hours, as compared to the immediate release dexamethasone (HEMADY®), where 40% of the API was released within 15 minutes in 0.01N HCl and plateaued at 95% after just 2 hours minutes.

### Example 7

Further, in Example 7 and referring to Table 15, the basic compositions for lenalidomide that were produced successfully and within current FDA inactive ingredient database guidelines are shown below, with the understanding that other ranges are contemplated by the present disclosure.

TABLE 15
Proposed Formulations of DEX Systems

| | Generic | % | Amount (mg/ |
|---|---|---|---|
| Ingredients | Name | Composition | tablet) |
| Dexamethasone | Dexamethasone | 0.2%-5% | 1 mg to 25 mg |
| Kollidon 90 | Povidone (PVP) | 5%-15% | 25 mg to |
| | | | 75 mg |
| Klucel EF | Hydroxypropyl | 5%-15% | 25 mg to |
| | cellulose (HPC) | | 75 mg |
| Carbopol 981 | Carbomer | 0.5%-15% | 25 mg to |
| | Homopolymer | | 75 mg |
| | Type A | | |
| Lactose | Lactose | Qs: 30%-86% | 150 mg to |
| | | | 410 mg |
| Parteck SLC | Silicon dioxide | 1%-10% | 5 mg to 50 mg |
| | (SiO2) | | |
| Kolliphor P407 | Poloxamer 407 | 1%-10% | 5 mg to 50 mg |

| | | |
|---|---|---|
| Target Tablet or Capsule | 500 mg | 500 mg |
| Fill Weight | | |

The API can be added into the dry powder blending process as one of the following:
1) Dexamethasone, micronized

It is contemplated that the composition of dexamethasone with HPC, PVP, Carbomer, Poloxamer 407, silicon dioxide and lactose in the ranges stated above will achieve:
1) A reduction in drug loading is feasible to achieve an equivalent AUC with reduced Cmax with a controlled steady and predictable release of drug from the tablet/capsule compared to the Hemady® or other immediate release tablets based on an in silico modeling. (See below)
2) A reduction in Cmax by about 90% from about 247 ng/mL from Hemady® 20 mg tablets to about 28 ng/mL from controlled release dosage form with only a 5 mg drug loading which is not taking into account the increase to match bioavailability to the 20 mg IR tablet. The potential for reduced exposure by total amount of 10 mg drug loaded into the dosage form is based on the in silico modeling.
3) An increase in Tmax from 2 hours with Hemady® to about 8-12 hours.

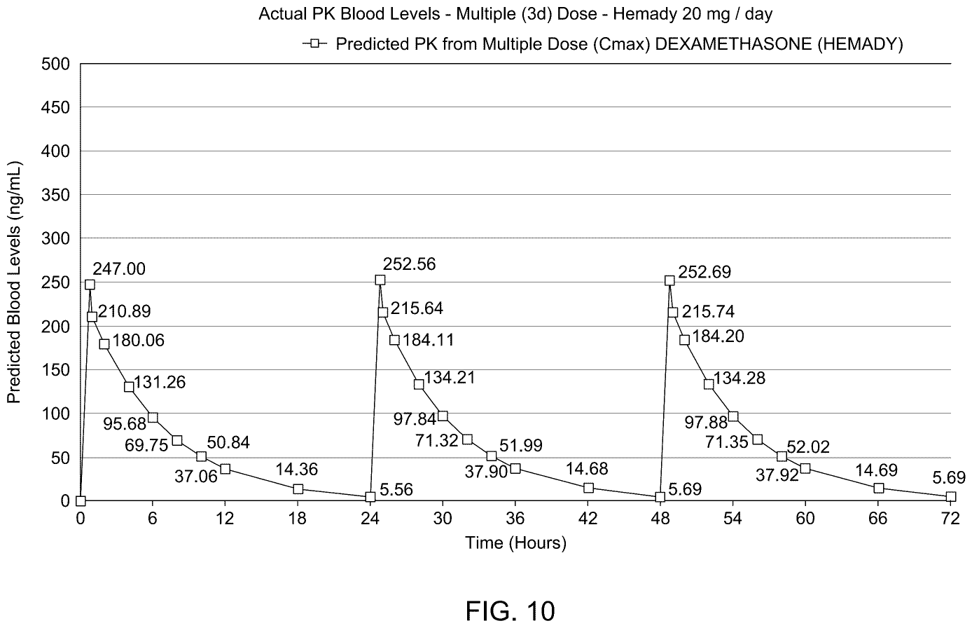

Further, referring to FIG. 10, the PK blood levels for multiple day dosing of Hemady® 20 mg in tablet form illustrate a profile resembles the typical dump-and-die profile associated with oral delivery of short-half-life APIs, an issue that the present disclosure solves.

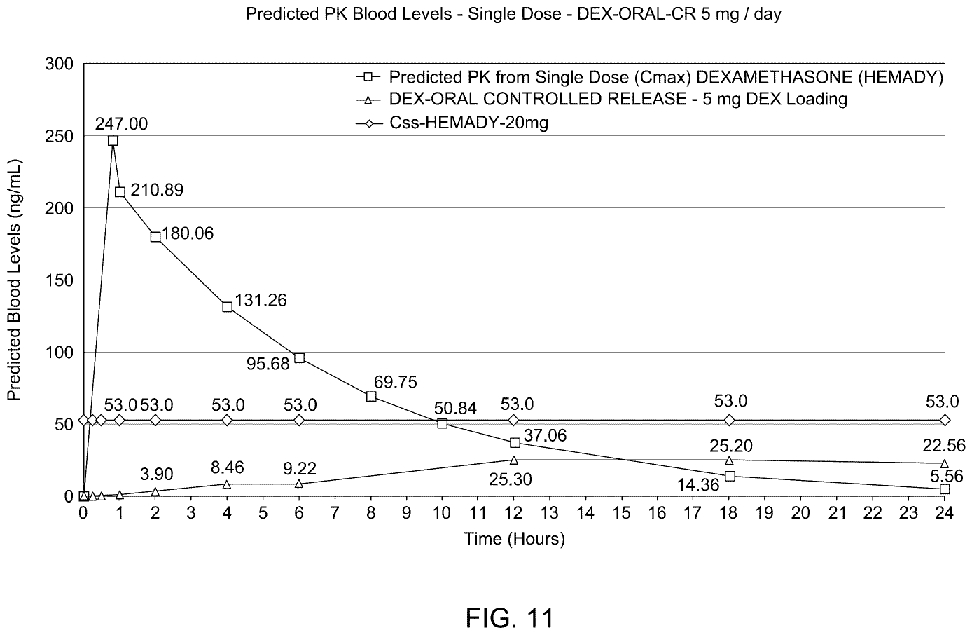

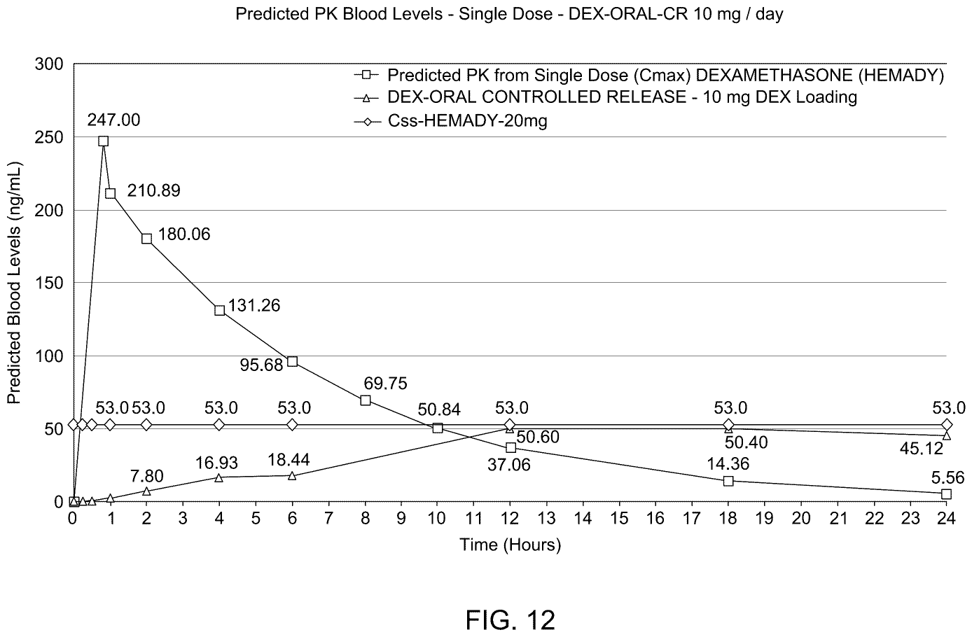

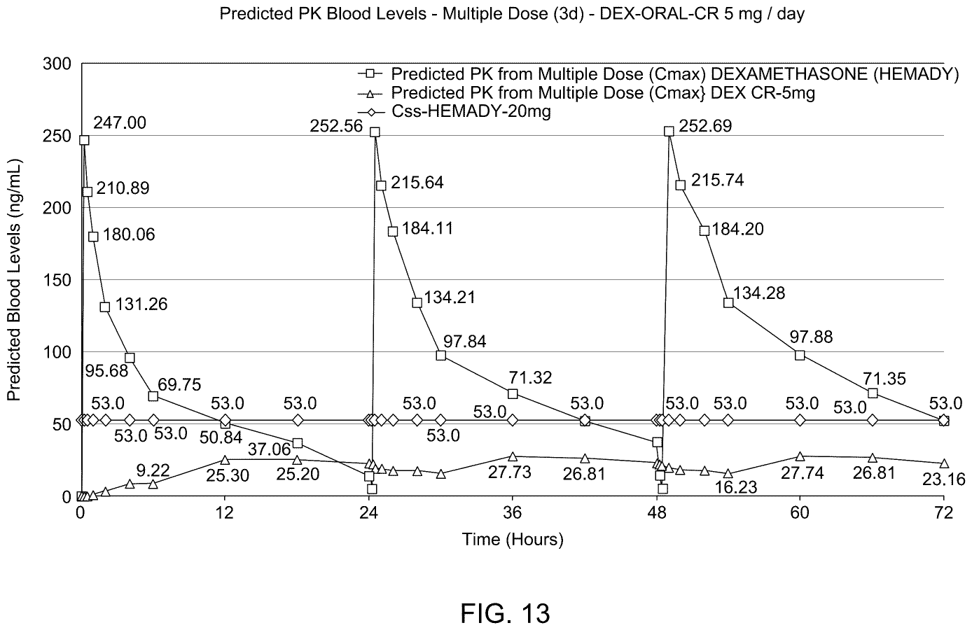

In particular, FIGS. 11-13 illustrate the PK blood levels of the controlled release solid oral dosage forms contemplated by the present disclosure compared to the immediate release dosage form with a target blood level calculated at 24 hours of about 53 ng/mL based on the Hemady® 20 mg dosage form suggesting the equivalent controlled release profile may be reduced by about 50-75%, such that about a 5 to 8 mg oral dose with this controlled release profile would be in line with the target blood level concentration of the 20 mg Hemady® immediate release tablet.

### Example 8

In Example 8, various formulations contemplated by the present disclosure were formed to explore the effect of lenalidomide in combination with HPC, K-90 and various other controlled release polymers, including different types of crosslinked polyacrylic acid polymers (Carbopol® 971P, 974P, Ultrez® 10), crosslinked copolymers of acrylic acid and a C10-30 alkyl acrylate comonomer (Carbopol® ETD 2020 and Pemulen® TR1), and anionic copolymers based on methacrylic acid and methyl methacrylate (Eudragit® L100, S100, and L100-55), as shown below in Table 16.

TABLE 16
Example 8 Formulation Components by Wt. %

| Ingredients | 1 | 2 | 3 | 4 | 5 | 6 | 7 | 8 |
|---|---|---|---|---|---|---|---|---|
| Lenalidomide | 1% | 1% | 1% | 1% | 1% | 1% | 1% | 1% |
| Lactose | 55% | 55% | 55% | 55% | 55% | 55% | 55% | 55% |
| SiO₂ | 9% | 9% | 9% | 9% | 9% | 9% | 9% | 9% |
| PVP | 10% | 10% | 10% | 10% | 10% | 10% | 10% | 10% |
| HPC | 10% | 10% | 10% | 10% | 10% | 10% | 10% | 10% |
| Poloxamer | 5% | 5% | 5% | 5% | 5% | 5% | 5% | 5% |
| Carbopol 971P | 10% | — | — | — | — | — | — | — |
| Carbopol 974P | — | 10% | — | — | — | — | — | — |
| Ultrez 10 | — | — | 10% | — | — | — | — | — |
| ETD 2020 | — | — | — | 10% | — | — | — | — |
| Pemulen TR1 | — | — | — | — | 10% | — | — | — |
| Eudragit L100 | — | — | — | — | — | 10% | — | — |
| Eudragit S100 | — | — | — | — | — | — | 10% | — |
| Eudragit L100-55 | — | — | — | — | — | — | — | 10% |

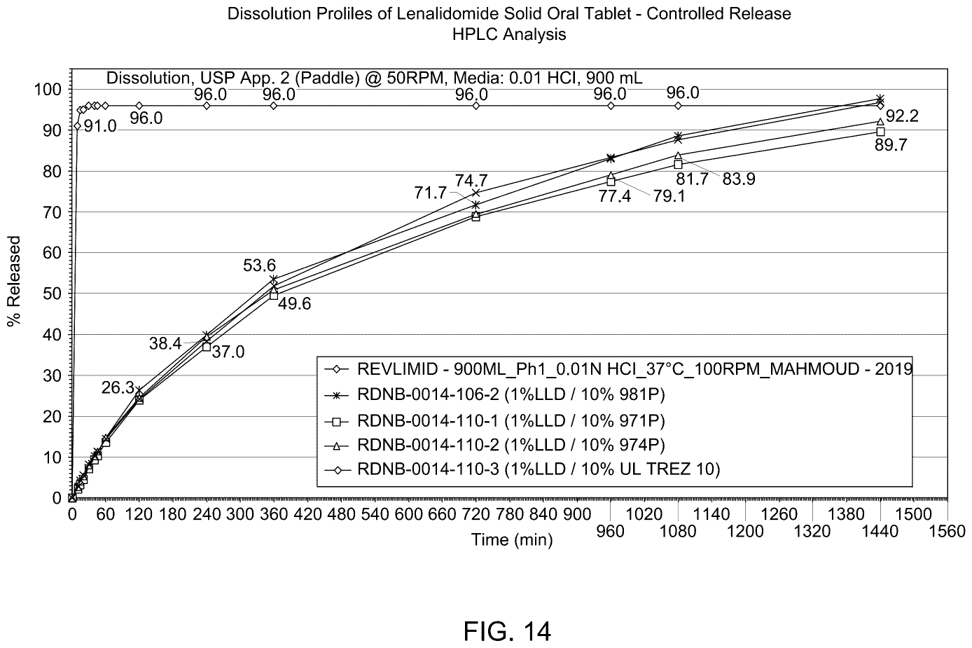

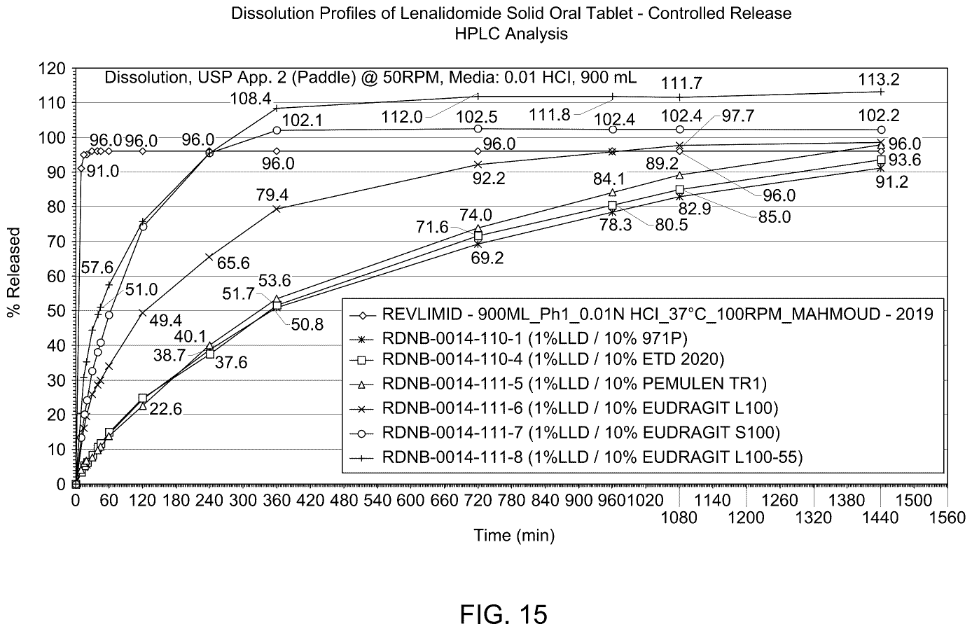

As shown in FIG. 14, the release profile of the crosslinked polyacrylic acid polymers in the form of Carbopol® 971P, 974P, Ultrez® 10 provide improved release profiles similar to that of Carbopol® 981P (see prior Examples), exhibiting a much more gradual, controlled release compared to the immediate release Revlimid® capsule. Further, referring to FIG. 15, the C10-30 alkyl acrylate comonomers in the form of Carbopol® ETD 2020 and Pemulen® TR1 also showed improved release profiles similar to that of Carbopol® 981P (see prior Examples), exhibiting a much more gradual, controlled release compared to the immediate release Revlimid® capsule. Controlled near zero-order release profiles are obtained for each composition. Meanwhile, the anionic copolymers based on methacrylic acid and methyl methacrylate in the form of Eudragit® L100, S100, and L100-55) do not provide as much improvement in the dissolution release rate and show double to triple the release rate at about 40 minutes to about 6 hours.

### Example 9

In Example 9, the effect of the concentration of Carbopol® 971P on the release profiles of the formulations contemplated by the present disclosure was examined.

TABLE 17
Example 9 Formulation Components by Wt. %

| Ingredients | 112-1 | 112-2 | 112-3 | 112-4 | 112-5 | 112-6 |
|---|---|---|---|---|---|---|
| Lenalidomide | 1% | 1% | 1% | 1% | 1% | 1% |
| Lactose | 60% | 55% | 50% | 45% | 40% | 15% |
| SiO₂ | 9% | 9% | 9% | 9% | 9% | 9% |
| PVP | 10% | 10% | 10% | 10% | 10% | 10% |
| Carbopol 971P | 5% | 10% | 15% | 20% | 25% | 50% |
| HPC | 10% | 10% | 10% | 10% | 10% | 10% |
| Poloxamer | 5% | 5% | 5% | 5% | 5% | 5% |

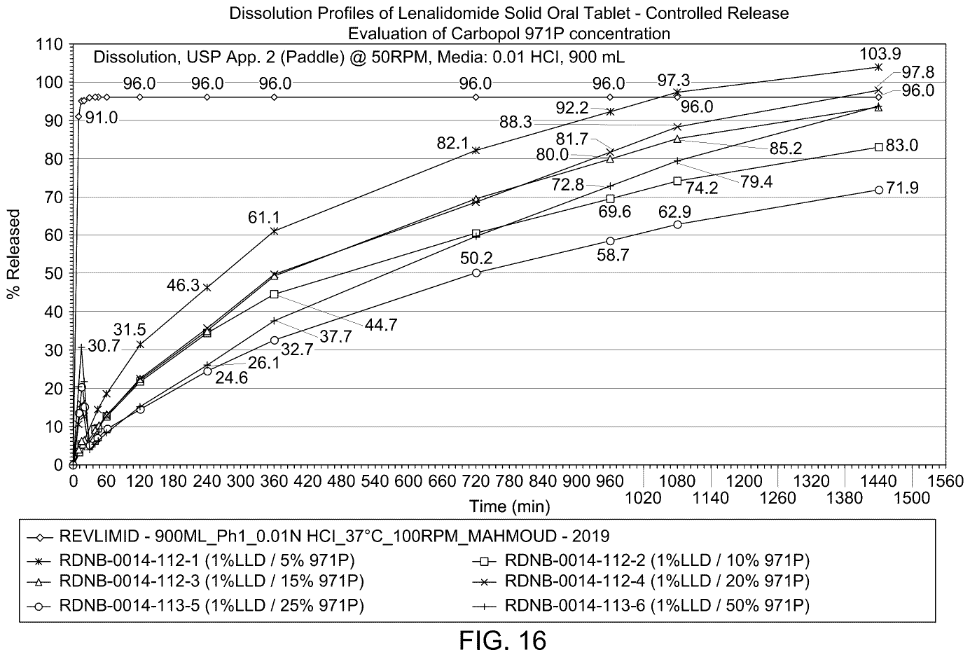

As shown in FIG. 16, various formulations were studied to determine the relationship between the amount of Carbopol® 971P present (with replacement by lactose to maintain the other excipients at the same concentrations) and the formulations release profiles. Based on the concentration of Carbopol® 971P used, preferred release rates are achieved from about 5 wt. % to about 50% wt. % Carbopol® 971P, such as from about 5 wt. % to about 25 wt. %, such as from about 10 wt. % to about 25 wt. %.

### Example 10

In Example 10, the effect of the concentration of poloxamer on the release profiles of the formulations contemplated by the present disclosure was examined.

TABLE 18
Example 10 Formulation Components by Wt. %

| Ingredients | 114-1 | 114-2 | 114-3 | 114-4 |
|---|---|---|---|---|
| Lenalidomide | 1% | 1% | 1% | 1% |
| Lactose | 42.5% | 40% | 37.5% | 35% |
| SiO₂ | 9% | 9% | 9% | 9% |
| PVP | 10% | 10% | 10% | 10% |
| Carbopol 971P | 25% | 25% | 25% | 25% |
| HPC | 10% | 10% | 10% | 10% |
| Poloxamer | 2.5% | 5% | 7.5% | 10% |

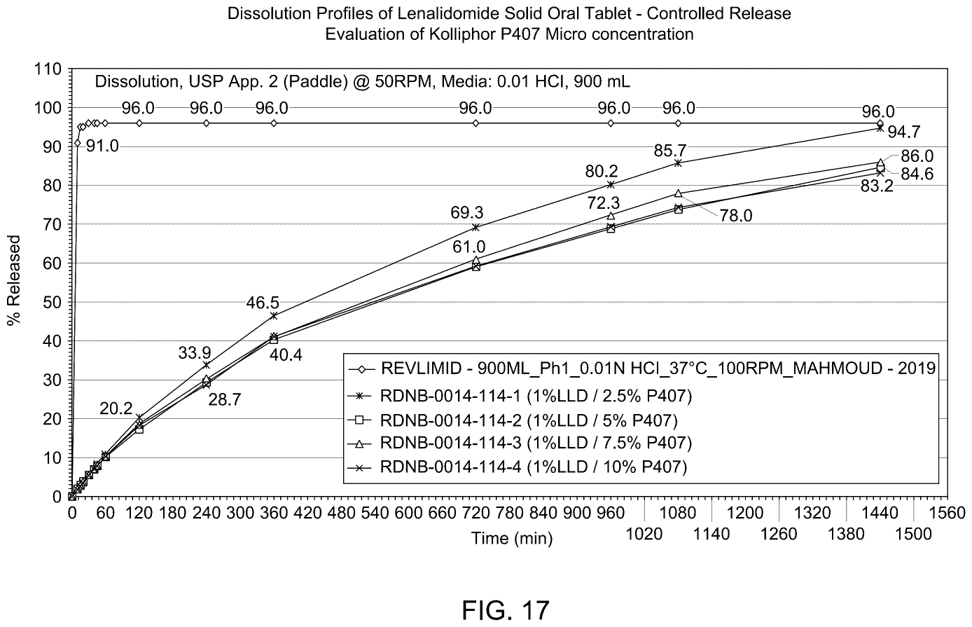

As shown in FIG. 17, although poloxamers have been marketed to promote controlled release, it was found that in the range of about 2.5 wt. % to about 10 wt. %, there is no significant difference in the release rate of the formulations shown in Table 18. Further, it was found that the incorporation of poloxamer P407 actually promotes the ability of the powder to flow and reduce sticking or picking of the formulation during tablet production, thus a range of poloxamer from about 2.5 wt. % to 10 wt. % as a tableting lubricant is useful in place of standard lubricating agents, such as magnesium stearate, sodium stearyl fumarate, talc, beeswax, polyethylene glycols, or others.

### Example 11

In Example 11, the controlled release polymer composition comprising 1% or 2% Lenalidomide was filled directly into HPMC capsule shells. The comparison of fill volume to the size of the HPMC capsule from Size 1, 00, and 000 was evaluated.

TABLE 19
Example 11 Formulation Components by Wt. %

| | 168-1 | 168-2 |
|---|---|---|
| Ingredients | Blend from 14-114-4 | Blend from 14-115-8 |
| Lenalidomide | 1% | 2% |
| Lactose | 35% | 34% |
| SiO₂ | 9% | 9% |
| PVP | 10% | 10% |
| Carbopol 971P | 25% | 25% |
| HPC | 10% | 10% |
| Poloxamer | 10% | 10% |
| Filled Amount in | 260 mg | 250 mg |
| HPMC Capsule Size 1 | | |
| Filled Amount in | 410 mg | 420 mg |
| HPMC Capsule Size 00 | | |
| Filled Amount in | 750 mg | 730 mg |
| HPMC Capsule Size 000 | | |

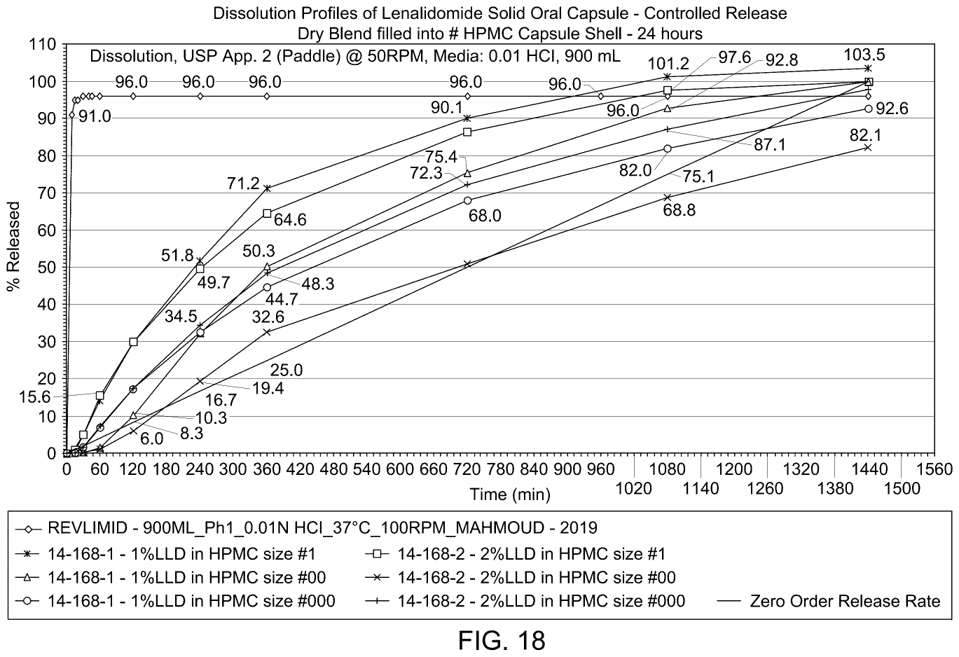

As shown in FIG. 18, it was found that the size of HPMC capsule (e.g., size 1, 00, or 000) can have a significant impact based on volume of controlled release dry powder and the hydration event which leads to the diffusion of the API from the wetted matrix. The smaller the capsule, the higher the rate of diffusion, and the larger the capsule, the slower the rate of diffusion from the matrix. Drug release from different % w/w loading of lenalidomide from 1 wt. % to 2 wt. % did not have a significant impact on the release of lenalidomide from the matrix.

### Example 12

In Example 12, the controlled release polymer composition comprising 2 wt. % lenalidomide was filled directly into standard HPMC capsule shells or enteric (HPMC/HPMC-P) capsule shells. The size 0 capsule and filled amount (about 360 mg to about 370 mg) was kept constant between the two (2) different capsule shells.

TABLE 20
Example 12 Formulation Components by Wt. %

| | HPMC | Enteric HPMC/HPMC-P |
|---|---|---|
| Ingredients | Capsule | Capsule |
| Lenalidomide | 2% | 2% |
| Lactose | 34% | 34% |
| SiO₂ | 9% | 9% |
| PVP | 10% | 10% |
| Carbopol 971P | 25% | 25% |
| HPC | 10% | 10% |
| Poloxamer | 10% | 10% |
| Capsule Size 0 | 340 mg | 365 mg |
| Filled Quantity | | |

As shown in FIG. 18, it was found that the type of capsule shell exhibits a significant contribution to the control of the release of the lenalidomide. Enteric capsule shells made from a blend of HPMC and HPMC-P control the release of lenalidomide from the matrix by more than 2-fold for the initial 8-12 hours.

While various embodiments of the present disclosure have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. Thus, the breadth and scope of the present disclosure should not be limited by any of the above-described exemplary embodiments.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the present disclosure, but instead as merely intended to highlight alternative or additional features that may or may not be utilized in a particular embodiment of the present disclosure. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments. For instance, when a plurality of ranges are provided, any combination of a minimum value and a maximum value described in the plurality of ranges are contemplated by the present disclosure. For example, if ranges of ‘from about 20% to about 80%’ and ‘from about 30% to about 70%’ are described, a range of ‘from about 20% to about 70%’ or a range of ‘from about 30% to about 80%’ are also contemplated by the present disclosure.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and ‘one or more’ to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more” or ‘at least one’ and indefinite articles such as ‘a’ or ‘an’ (e.g., ‘a’ and/or ‘an’ should typically be interpreted to mean ‘at least one’ or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the terms ‘about,’ ‘approximately,’ or ‘generally.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches. As used herein, the terms ‘about,’ ‘approximately,’ or ‘generally,’ when used to modify a value, indicate that the value can be raised or lowered by 5% and remain within the disclosed embodiment.

All of the features disclosed in this specification (including any accompanying exhibits, claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure covers such alterations, variations, and equivalents.

What is claimed is:

1. A formulation for oral administration, the formulation comprising:
an active pharmaceutical ingredient (API) having a water solubility of less than about 10 mg/mL and a melting point of greater than about 120° C., wherein the API is present in the formulation in an amount ranging from about 0.05 wt. % to about 10 wt. % based on the total weight of the formulation;
a carrier powder, wherein the carrier powder is present in the formulation in an amount ranging from about 0.5 wt. % to about 15 wt. % based on the total weight of the formulation;
a filler, wherein the filler is present in the formulation in an amount ranging from about 15 wt. % to about 85 wt. % based on the total weight of the formulation; and
a polymer system comprising one or more controlled release polymers, wherein the polymer system is present in the formulation in an amount ranging from about 10 wt. % to about 70 wt. % based on the total weight of the formulation.

2. The formulation of claim 1, wherein the API comprises an immunomodulatory imide compound, a Cereblon E3 ligase modulator (CELMoD), a steroid, or a combination thereof.

3. The formulation of claim 2, wherein the immunomodulatory imide compound comprises lenalidomide, pomalidomide, iberdomide, or a combination thereof, the Cereblon E3 ligase modulator (CELMoD) comprises mezigdomide, golcadomide, or a combination thereof, and wherein the steroid comprises dexamethasone, dexamethasone acetate, dexamethasone sodium phosphate, betamethasone, or a combination thereof.

4. The formulation of claim 2, wherein the one or more APIs comprises lenalidomide and dexamethasone.

5. The formulation of claim 2, wherein the one or more APIs comprises pomalidomide and dexamethasone.

6. The formulation of claim 1, wherein the carrier powder comprises silicon dioxide, polyvinylpyrrolidone, or a combination thereof.

7. The formulation of claim 6, wherein the carrier powder comprises silicone dioxide, wherein the API is present in the carrier powder at a weight ratio of the API to the carrier powder ranging from 1:1.25 to about 1:15.

8. The formulation of claim 1, wherein the carrier powder comprises polyvinylpyrrolidone, wherein the API is present in the carrier powder at a weight ratio of the API to the carrier powder ranging from about to about 1:0.5 to about 1:2.

9. The formulation of claim 1, wherein the polymer system comprises polyvinylpyrrolidone, hydroxypropyl cellulose, crosslinked polyacrylic acid, or a combination thereof.

10. The formulation of claim 1, wherein the formulation further comprises a poloxamer, wherein the poloxamer is present in the formulation in an amount ranging from about 0.5 wt. % to about 15 wt. % based on the total weight of the formulation.

11. The formulation of claim 1, wherein the formulation is surrounded by an enteric coating or is contained within an enteric capsule shell.

12. The formulation of claim 1, wherein the API comprises an immunomodulatory imide compound, wherein the API exhibits a percent release in 0.01N HCl ranging from about 1% to about 20% after about 10 minutes and a percent release in 0.01 N HCl ranging from about 70% to about 100% after about 24 hours.

13. The formulation of claim 1, wherein the API comprises a steroid, wherein the API exhibits a percent release in 0.01 N HCl ranging from about 0.5% to about 15% after about 15 minutes and a percent release in 0.01 N HCl ranging from about 35% to about 100% after about 24 hours.

14. A method of making a formulation for oral administration, the method comprising:
combining an active pharmaceutical ingredient (API) having a water solubility of less than about 10 mg/mL and a melting point of greater than about 120° C. with a carrier powder, a filler, and a polymer system comprising one or more controlled release polymers to form a dry powder blend.

15. The method of claim 14, wherein the polymer system comprises a polyvinylpyrrolidone, a hydroxypropyl cellulose, a crosslinked polyacrylic acid, or a combination thereof.

16. The method of claim 14, further comprising compressing the dry powder blend into tablet form.

17. The method of claim 14, wherein the formulation is surrounded by an enteric coating or is contained within an enteric capsule shell.

18. A method of delivering a formulation to a subject, the method comprising:
administering the formulation to the subject orally,
wherein the formulation comprises:
an active pharmaceutical ingredient (API) having a water solubility of less than about 10 mg/mL and a melting point of greater than about 120° C., wherein the API is present in the formulation in an amount ranging from about 0.05 wt. % to about 10 wt. % based on the total weight of the formulation;
a carrier powder, wherein the carrier powder is present in the formulation in an amount ranging from about 0.5 wt. % to about 15 wt. % based on the total weight of the formulation;
a filler, wherein the filler is present in the formulation in an amount ranging from about 15 wt. % to about 85 wt. % based on the total weight of the formulation; and
a polymer system comprising one or more controlled release polymers, wherein the polymer system is present in the formulation in an amount ranging from about 10 wt. % to about 70 wt. % based on the total weight of the formulation.

19. The method of claim 18, wherein the formulation is administered via a tablet or capsule.

20. The method of claim 18, wherein the formulation is surrounded by an enteric coating or is contained within an enteric capsule shell.
